SEQUENCE LISTING (10 sequences)

SEQ ID NO: 1 - DNA, 1698 bp, Artificial Sequence
  Mutant P22sTsp5H-x; head-to-head hexamer configuration
atgtgccatcaccatcaccatcacgatccagatcaatattcaatagaagctgataaaaaatttaagtattcagtaaaattatcagattatccaacattgcaggatgcagcatctgctgcggttgatggccttcttatcgatcgagattataatttttatggtggagagacagttgattttggcggaaaggttctgactatagaatgtaaagctaaatttataggagatggaaatcttatttttacgaaattaggcaaaggttcccgcattgccggggtttttatggaaagcactacaacaccatgggttatcaagccttggacggatgacaatcagtggctaacggatgccgcagcggtcgttgccactttaaaacaatctaaaactgatgggtatcagccaaccgtaagcgattacgttaaattcccaggaatagaaacgttactcccacctaatgcaaaagggcaaaacataacgtctacgttagaaattagagaatgtataggggtcgaagttcatcgggctagcggtctaatggctggttttttgtttagagggtgtcacttctgcaagatggtagacgccaataatccaagcggaggtaaagatggcattataaccttcgaaaaccttagcggcgattgggggaagggtaactatgtcattggcggacgaaccagctatgggtcagtaagtagcgcccagtttttacgtaataatggtggctttgaacgtgatggtggagttattgggtttacttcatatcgcgctggggagagtggcgttaaaacttggcaaggtactgtgggctcgacaacctctcgcaactataatctgcaattccgcgactcggtcgttatttaccccgtatggaatggattcgatttaggtgctgacactgacatgaatccggagttggacaggccaggggactaccctataacccaatacccactgcatcagttacccctaaatcacctgattgataatcttctggttcgcggggcgttaggtgtaggttttggtatggatggtaagggcatgtatgtgtctaatattaccgtagaagattgcgctggctctggcgcgtacctactcacccatgaatcagtatttaccaatatagccataattgataccaatactaagggtttccaggctaatcagatttatatatctggggcttgccgtgtgaacggtttacgtttaattgggatccgctcaaccgatgggcagggtctaaccatagacgcccctaactctaccgtaagcggtatcaccgggatggtagacccctctagaattaatgttgctaatttggcagaagaagggttaggtaatatccgcgctaatagtttcggctatgatagcgcagcgattagactgcggattcataagttatcaaagaccttagatagcggagcattgtactcccacattaacgtggggcccggttctggctcagcgtggactcaacttactgctatttcaggtaacacacctgacgctgtatcattaaaagttaaccacaaagattgcaggggggcagagataccatttgtccctgacatcgcgtcagatgattttataaaggattcctcatgttttttgccatattgggaaaataattctacttctttaaaggctttagtgaaaaaacccaatggagaattagttagattaaccttggcaacactt

SEQ ID NO: 2 - protein, 566 aa, Artificial Sequence
  Protein encoded by SEQ ID NO1 (P22sTsp5H-x)
MCHHHHHHDPDQYSIEADKKFKYSVKLSDYPTLQDAASAAVDGLLIDRDYNFYGGETVDFGGKVLTIECKAKFIGDGNLIFTKLGKGSRIAGVFMESTTTPWVIKPWTDDNQWLTDAAAVVATLKQSKTDGYQPTVSDYVKFPGIETLLPPNAKGQNITSTLEIRECIGVEVHRASGLMAGFLFRGCHFCKMVDANNPSGGKDGIITFENLSGDWGKGNYVIGGRTSYGSVSSAQFLRNNGGFERDGGVIGFTSYRAGESGVKTWQGTVGSTTSRNYNLQFRDSVVIYPVWNGFDLGADTDMNPELDRPGDYPITQYPLHQLPLNHLIDNLLVRGALGVGFGMDGKGMYVSNITVEDCAGSGAYLLTHESVFTNIAIIDTNTKGFQANQIYISGACRVNGLRLIGIRSTDGQGLTIDAPNSTVSGITGMVDPSRINVANLAEEGLGNIRANSFGYDSAAIRLRIHKLSKTLDSGALYSHINVGPGSGSAWTQLTAISGNTPDAVSLKVNHKDCRGAEIPFVPDIASDDFIKDSSCFLPYWENNSTSLKALVKKPNGELVRLTLATL

SEQ ID NO: 3 - DNA, 1707 bp, Artificial Sequence
  mutant P22sTsp5-x; tail-to-tail hexamer configuration
atgcatcaccatcaccatcacgatccagatcaatattcaatagaagctgataaaaaatttaagtattcagtaaaattatcagattatccaacattgcaggatgcagcatctgctgcggttgatggccttcttatcgatcgagattataatttttatggtggagagacagttgattttggcggaaaggttctgactatagaatgtaaagctaaatttataggagatggaaatcttatttttacgaaattaggcaaaggttcccgcattgccggggtttttatggaaagcactacaacaccatgggttatcaagccttggacggatgacaatcagtggctaacggatgccgcagcggtcgttgccactttaaaacaatctaaaactgatgggtatcagccaaccgtaagcgattacgttaaattcccaggaatagaaacgttactcccacctaatgcaaaagggcaaaacataacgtctacgttagaaattagagaatgtataggggtcgaagttcatcgggctagcggtctaatggctggttttttgtttagagggtgtcacttctgcaagatggtagacgccaataatccaagcggaggtaaagatggcattataaccttcgaaaaccttagcggcgattgggggaagggtaactatgtcattggcggacgaaccagctatgggtcagtaagtagcgcccagtttttacgtaataatggtggctttgaacgtgatggtggagttattgggtttacttcatatcgcgctggggagagtggcgttaaaacttggcaaggtactgtgggctcgacaacctctcgcaactataatctgcaattccgcgactcggtcgttatttaccccgtatggaatggattcgatttaggtgctgacactgacatgaatccggagttggacaggccaggggactaccctataacccaatacccactgcatcagttacccctaaatcacctgattgataatcttctggttcgcggggcgttaggtgtaggttttggtatggatggtaagggcatgtatgtgtctaatattaccgtagaagattgcgctggctctggcgcgtacctactcacccatgaatcagtatttaccaatatagccataattgataccaatactaagggtttccaggctaatcagatttatatatctggggcttgccgtgtgaacggtttacgtttaattgggatccgctcaaccgatgggcagggtctaaccatagacgcccctaactctaccgtaagcggtatcaccgggatggtagacccctctagaattaatgttgctaatttggcagaagaagggttaggtaatatccgcgctaatagtttcggctatgatagcgcagcgattagactgtggattcataagttatcaaagaccttagatagcggagcattgtactcccacattaacgtggggcccggttctggctcagcgtggactcaacttactgctatttcaggtaacacacctgacgctgtatcattaaaagttaaccacaaagattgcaggggggcagagataccatttgtccctgacatcgcgtcagatgattttataaaggattcctcatgttttttgccatattgggaaaataattctacttctttaaaggctttagtgaaaaaacccaatggagaattagttagattaaccttggcaacacttagatccggctgc

SEQ ID NO: 4 - protein, 569 aa, Artificial Sequence
  Protein encoded by SEQ ID NO3; P22sTsp5-x
MHHHHHHDPDQYSIEADKKFKYSVKLSDYPTLQDAASAAVDGLLIDRDYNFYGGETVDFGGKVLTIECKAKFIGDGNLIFTKLGKGSRIAGVFMESTTTPWVIKPWTDDNQWLTDAAAVVATLKQSKTDGYQPTVSDYVKFPGIETLLPPNAKGQNITSTLEIRECIGVEVHRASGLMAGFLFRGCHFCKMVDANNPSGGKDGIITFENLSGDWGKGNYVIGGRTSYGSVSSAQFLRNNGGFERDGGVIGFTSYRAGESGVKTWQGTVGSTTSRNYNLQFRDSVVIYPVWNGFDLGADTDMNPELDRPGDYPITQYPLHQLPLNHLIDNLLVRGALGVGFGMDGKGMYVSNITVEDCAGSGAYLLTHESVFTNIAIIDTNTKGFQANQIYISGACRVNGLRLIGIRSTDGQGLTIDAPNSTVSGITGMVDPSRINVANLAEEGLGNIRANSFGYDSAAIRLWIHKLSKTLDSGALYSHINVGPGSGSAWTQLTAISGNTPDAVSLKVNHKDCRGAEIPFVPDIASDDFIKDSSCFLPYWENNSTSLKALVKKPNGELVRLTLATLRSGC

SEQ ID NO: 5 - DNA, 1698 bp, Artificial Sequence
  HEAD-TO-HEAD HEXAMER (P22sTsp5)
atgtgccatcaccatcaccatcacgatccagatcaatattcaatagaagctgataaaaaatttaagtattcagtaaaattatcagattatccaacattgcaggatgcagcatctgctgcggttgatggccttcttatcgatcgagattataatttttatggtggagagacagttgattttggcggaaaggttctgactatagaatgtaaagctaaatttataggagatggaaatcttatttttacgaaattaggcaaaggttcccgcattgccggggtttttatggaaagcactacaacaccatgggttatcaagccttggacggatgacaatcagtggctaacggatgccgcagcggtcgttgccactttaaaacaatctaaaactgatgggtatcagccaaccgtaagcgattacgttaaattcccaggaatagaaacgttactcccacctaatgcaaaagggcaaaacataacgtctacgttagaaattagagaatgtataggggtcgaagttcatcgggctagcggtctaatggctggttttttgtttagagggtgtcacttctgcaagatggtagacgccaataatccaagcggaggtaaagatggcattataaccttcgaaaaccttagcggcgattgggggaagggtaactatgtcattggcggacgaaccagctatgggtcagtaagtagcgcccagtttttacgtaataatggtggctttgaacgtgatggtggagttattgggtttacttcatatcgcgctggggagagtggcgttaaaacttggcaaggtactgtgggctcgacaacctctcgcaactataatctgcaattccgcgactcggtcgttatttaccccgtatgggacggattcgatttaggtgctgacactgacatgaatccggagttggacaggccaggggactaccctataacccaatacccactgcatcagttacccctaaatcacctgattgataatcttctggttcgcggggcgttaggtgtaggttttggtatggatggtaagggcatgtatgtgtctaatattaccgtagaagattgcgctggctctggcgcgtacctactcacccatgaatcagtatttaccaatatagccataattgataccaatactaagggtttccaggctaatcagatttatatatctggggcttgccgtgtgaacggtttacgtttaattgggatccgctcaaccgatgggcagggtctaaccatagacgcccctaactctaccgtaagcggtatcaccgggatggtagacccctctagaattaatgttgctaatttggcagaagaagggttaggtaatatccgcgctaatagtttcggctatgatagcgcagcgattagactgcggattcataagttatcaaagaccttagatagcggagcattgtactcccacattaacgtggggcccggttctggctcagcgtggactcaacttactgctatttcaggtaacacacctgacgctgtatcattaaaagttaaccacaaagattgcaggggggcagagataccatttgtccctgacatcgcgtcagatgattttataaaggattcctcatgttttttgccatattgggaaaataattctacttctttaaaggctttagtgaaaaaacccaatggagaattagttagattaaccttggcaacactt

SEQ ID NO: 6 - protein, 566 aa, Artificial Sequence
  Protein ecnoded by SEQ ID NO5 (P22sTsp5)
MCHHHHHHDPDQYSIEADKKFKYSVKLSDYPTLQDAASAAVDGLLIDRDYNFYGGETVDFGGKVLTIECKAKFIGDGNLIFTKLGKGSRIAGVFMESTTTPWVIKPWTDDNQWLTDAAAVVATLKQSKTDGYQPTVSDYVKFPGIETLLPPNAKGQNITSTLEIRECIGVEVHRASGLMAGFLFRGCHFCKMVDANNPSGGKDGIITFENLSGDWGKGNYVIGGRTSYGSVSSAQFLRNNGGFERDGGVIGFTSYRAGESGVKTWQGTVGSTTSRNYNLQFRDSVVIYPVWDGFDLGADTDMNPELDRPGDYPITQYPLHQLPLNHLIDNLLVRGALGVGFGMDGKGMYVSNITVEDCAGSGAYLLTHESVFTNIAIIDTNTKGFQANQIYISGACRVNGLRLIGIRSTDGQGLTIDAPNSTVSGITGMVDPSRINVANLAEEGLGNIRANSFGYDSAAIRLRIHKLSKTLDSGALYSHINVGPGSGSAWTQLTAISGNTPDAVSLKVNHKDCRGAEIPFVPDIASDDFIKDSSCFLPYWENNSTSLKALVKKPNGELVRLTLATL

SEQ ID NO: 7 - DNA, 1709 bp, Artificial Sequence
  Nucleotide encoding P22sTsp5 (tail-to-tail hexamer)
atgcatcaccatcaccatcacgatccagatcaatattcaatagaagctgataaaaaatttaagtattcagtaaaattatcagattatccaacattgcaggatgcagcatctgctgcggttgatggccttcttatcgatcgagattataatttttatggtggagagacagttgattttggcggaaaggttctgactatagaatgtaaagctaaatttataggagatggaaatcttatttttacgaaattaggcaaaggttcccgcattgccggggtttttatggaaagcactacaacaccatgggttatcaagccttggacggatgacaatcagtggctaacggatgccgcagcggtcgttgccactttaaaacaatctaaaactgatgggtatcagccaaccgtaagcgattacgttaaattcccaggaatagaaacgttactcccacctaatgcaaaagggcaaaacataacgtctacgttagaaattagagaatgtataggggtcgaagttcatcgggctagcggtctaatggctggttttttgtttagagggtgtcacttctgcaagatggtagacgccaataatccaagcggaggtaaagatggcattataaccttcgaaaaccttagcggcgattgggggaagggtaactatgtcattggcggacgaaccagctatgggtcagtaagtagcgcccagtttttacgtaataatggtggctttgaacgtgatggtggagttattgggtttacttcatatcgcgctggggagagtggcgttaaaacttggcaaggtactgtgggctcgacaacctctcgcaactataatctgcaattccgcgactcggtcgttatttaccccgtatgggacggattcgatttaggtgctgacactgacatgaatccggagttggacaggccaggggactaccctataacccaatacccactgcatcagttacccctaaatcacctgattgataatcttctggttcgcggggcgttaggtgtaggttttggtatggatggtaagggcatgtatgtgtctaatattaccgtagaagattgcgctggctctggcgcgtacctactcacccatgaatcagtatttaccaatatagccataattgataccaatactaagggtttccaggctaatcagatttatatatctggggcttgccgtgtgaacggtttacgtttaattgggatccgctcaaccgatgggcagggtctaaccatagacgcccctaactctaccgtaagcggtatcaccgggatggtagacccctctagaattaatgttgctaatttggcagaagaagggttaggtaatatccgcgctaatagtttcggctatgatagcgcagcgattagactgcggattcataagttatcaaagaccttagatagcggagcattgtactcccacattaacgtggggcccggttctggctcagcgtggactcaacttactgctatttcaggtaacacacctgacgctgtatcattaaaagttaaccacaaagattgcaggggggcagagataccatttgtccctgacatcgcgtcagatgattttataaaggattcctcatgttttttgccatattgggaaaataattctacttctttaaaggctttagtgaaaaaacccaatggagaattagttagattaaccttggcaacacttagatccggctgcta

SEQ ID NO: 8 - protein, 569 aa, Artificial Sequence
  Protein encoded by SEQ ID NO7 (P22sTsp5)
MHHHHHHDPDQYSIEADKKFKYSVKLSDYPTLQDAASAAVDGLLIDRDYNFYGGETVDFGGKVLTIECKAKFIGDGNLIFTKLGKGSRIAGVFMESTTTPWVIKPWTDDNQWLTDAAAVVATLKQSKTDGYQPTVSDYVKFPGIETLLPPNAKGQNITSTLEIRECIGVEVHRASGLMAGFLFRGCHFCKMVDANNPSGGKDGIITFENLSGDWGKGNYVIGGRTSYGSVSSAQFLRNNGGFERDGGVIGFTSYRAGESGVKTWQGTVGSTTSRNYNLQFRDSVVIYPVWDGFDLGADTDMNPELDRPGDYPITQYPLHQLPLNHLIDNLLVRGALGVGFGMDGKGMYVSNITVEDCAGSGAYLLTHESVFTNIAIIDTNTKGFQANQIYISGACRVNGLRLIGIRSTDGQGLTIDAPNSTVSGITGMVDPSRINVANLAEEGLGNIRANSFGYDSAAIRLRIHKLSKTLDSGALYSHINVGPGSGSAWTQLTAISGNTPDAVSLKVNHKDCRGAEIPFVPDIASDDFIKDSSCFLPYWENNSTSLKALVKKPNGELVRLTLATLRSGC

SEQ ID NO: 9 - protein, 667 aa, Artificial Sequence
  tail spike protein from Enterobacteria phage P22
MTDITANVVVSNPRPIFTESRSFKAVANGKIYIGQIDTDPVNPANQIPVYIENEDGSHVQITQPLIINAAGKIVYNGQLVKIVTVQGHSMAIYDANGSQVDYIANVLKYDPDQYSIEADKKFKYSVKLSDYPTLQDAASAAVDGLLIDRDYNFYGGETVDFGGKVLTIECKAKFIGDGNLIFTKLGKGSRIAGVFMESTTTPWVIKPWTDDNQWLTDAAAVVATLKQSKTDGYQPTVSDYVKFPGIETLLPPNAKGQNITSTLEIRECIGVEVHRASGLMAGFLFRGCHFCKMVDANNPSGGKDGIITFENLSGDWGKGNYVIGGRTSYGSVSSAQFLRNNGGFERDGGVIGFTSYRAGESGVKTWQGTVGSTTSRNYNLQFRDSVVIYPVWDGFDLGADTDMNPELDRPGDYPITQYPLHQLPLNHLIDNLLVRGALGVGFGMDGKGMYVSNITVEDCAGSGAYLLTHESVFTNIAIIDTNTKDFQANQIYISGACRVNGLRLIGIRSTDGQGLTIDAPNSTVSGITGMVDPSRINVANLAEEGLGNIRANSFGYDSAAIKLRIHKLSKTLDSGALYSHINGGAGSGSAYTQLTAISGSTPDAVSLKVNHKDCRGAEIPFVPDIASDDFIKDSSCFLPYWENNSTSLKALVKKPNGELVRLTLATL

SEQ ID NO: 10 - protein, 667 aa, Artificial Sequence
  tail spike protein of Enterobacteria phage ST64T
MTDITANVVVSNPRPIFTESRSFKAVANGKIYIGQIDTDPVNPANQIPVYIENEDGSHVQIAQPLIINAAGKIVYNGQLVKIVTVQGHSMAIYDANGSQVDYIANVLKYDPDQYSIEADKKFKYSVKLSDYPTLQDAASAAVDGLLIDVDYHFYNGEKVDFGGKVLTIECKAKFIGDGNLIFTKLGKGSRIAGVFMESTTTPWVIKPWTDDNQWLTDAAAVVATLKQSKTDGYQPTVSDYVKFPGIETLLPPNAKGQNITSTLEIRECIGVEVHRASGLMAGFLFRGCHFCKMVDANNPSGGKDGIITFENLSGDWGKGNYVIGGRTSYGSVSSAQFLRNNGGFERDGGVIGFTSYRAGESGVKTWQGTVGSTTSRNYNLQFRDSVVIYPVWDGFDLGADTDMNPELDRPGDYPITQYPLHQLPLNHLIDNLLVRGALGVGFGMDGKGMYVSNITVEDCAGSGAYLLTHESVFTNIAIIDTNTKDFQANQIYISGACRVNGLRLIGIRSTDGQGLTIDAPNSTVSGITGMVDPSRINVANLAEEGLGNIRANSFGYDSAAIKLRIHKLSKTLDSGALYSHINVGPGSGSAWTQLTAISGNTPDAVSLKVNHKDCRGAEIPFVPDIASDDFIKDSSCFLPYWENNSTSLKALVKKPNGELVRLTLATL

(19) United States
(12) Patent Application Publication
(10) Pub. No.: US 2011/0143997 A1
(43) Pub. Date: Jun. 16, 2011

(54) PHAGE RECEPTOR BINDING PROTEINS FOR ANTIBACTERIAL THERAPY AND OTHER NOVEL USES

(75) Inventors: Matthew J. Henry, Indianapolis, IN (US); Roger C. Mackenzie, Ottawa (CA); Christine Szymanski, Ottawa (CA); Jamshid Tanha, Ottawa (CA)

(73) Assignees: Dow AgroSciences LLC, Indianapolis (IN); National Research Council of Canada, Ottawa, ON (CA)

(21) Appl. No.: 12/593,949

(22) PCT Filed: Mar. 28, 2008
(86) PCT No.: PCT/US08/58675

§ 371 Date: Jan. 19, 2010

Related U.S. Application Data
(60) Provisional application No. 60/909,044, filed on Mar. 30, 2007.

(51) Int. Cl. A61K 38/16; A61P 31/04; A01N 37/18; C07K 14/005; A01P 1/00

(52) U.S. Cl. 514/2.4; 514/1.1; 514/21.2; 530/350

(57) ABSTRACT

The subject invention relates in part to novel uses of bacteriophage tail spike proteins (TSPs). Some preferred uses are therapeutic uses in animals, such as chickens, against pathogenic bacteria, such as Salmonella. Fragments of the TSPs can also be used according to the subject invention, particularly protein fragments comprising the phage receptor binding domains (PRBDs), which recognize their hosts and facilitate infection. The binding domains are specific to unique surface structures on bacteria and may be used for a variety of applications according to the subject invention. We have shown that by utilizing these PRBDs, it is possible to exploit the long-established evolutionary relationship between bacteria and their viruses (ie bacteriophages) that specifically infect them. The subject invention also relates in part to novel, synthetic forms of tail spike proteins. In some preferred embodiments, these are hexamers.

### BACKGROUND OF THE INVENTION

There is increasing public concern for food and water safety. In North America alone food and water contamination with Campylobacter, Salmonella and E. coli species results in millions of infections, tens of thousands of hospitalizations, hundreds of deaths, and economic cost in the billions of dollars. With the increased antibiotic resistance in bacteria and the decreasing use of antibiotics worldwide, there exists a need for novel approaches.

A revival of bacteriophage research has promoted the application of live bacteriophages for the prevention and reduction of pathogens (Atterbury & Connerton, AEM, August 2005; Curtin J J & Donlan R M, Antimicrob Agents Chemother, April 2006; Higgins, J P et al., Poult Sci., July 2005; Park S C & Nakai T., Dis Aquat Organ., Janaury 2003), as well as the characterization of phage genomes (Vander Byl C & Kropinski, J. Bac., November 2000) and phage receptor binding domains (Steinbacher S et al., PNAS, October 1996).

Published PCT Application WO 00/32825 teaches methods for developing novel anti-microbial agents based on bacteriophage genomics.

The tail fibers of Salmonella phage P22 comprise trimers of the tail spike protein. See FIG. 9. The 3D atomic structure of these proteins is known. Each monomer will bind a specific sugar molecule (on the surface of the Salmonella bacteria). Each tail spike protein comprises a region known as a phage receptor binding domain (PRBD), which binds the sugar. (Steinbacher S et al, PNAS, October 96). Some other similarly arranged phage and proteins are known.

Phage have been used to serotype bacteria because of their specific binding properties. Whole phage have been used for some antibacterial therapies. For example, Clark et al. provides a review on the usage of phage including in therapy and detection and typing of bacteria. Clark et al.; “Bacteriophages and biotechnology: vaccines, gene therapy and antibacterials”; Trends Biotechnol. 2006 May; 24(5):212-8. Epub 2006 Mar. 29. Review.

Fischetti et al. compares whole phage and phage components (lysins) for therapy. “Reinventing phage therapy: are the parts greater than the sum?” Nat Biotechnol. 2006 December; 24(12):1508-11.

Oral administration of phage-impregnated feed to ayu fish (Plecoglossus altivelis) resulted in protection against experimental infection with P. plecoglossicida. After oral administration of P. plecoglossicida, cells of this bacterium were always detected in the kidneys of control fish that did not receive the phage treatment, while the cells quickly disappeared from the phage-treated fish. Park, S. C., Shimamura, I., Fukunaga, M., Mori, K. I., and Nakai T.; “Isolation of bacteriophages specific to a fish pathogen, Pseudomonas plecoglossicida, as a candidate for disease control” (2000) Appl. Environ. Microbiol. 66: 1416-1422. Intact bacteriophage cocktails have been added to ready-to-eat meats and poultry products to protect consumers from L. monocytogenes. L H Lang, “FDA approves use of bacteriophages to be added to meat and poultry products”. Gastroenterology 131 (2006).

Tail spike proteins and fragments thereof have never heretofore been used therapeutically.

### BRIEF SUMMARY OF THE INVENTION

The subject invention relates in part to novel uses of bacteriophage tail spike proteins (TSPs). Some preferred uses are therapeutic uses in animals, such as chickens, against pathogenic bacteria, such as Salmonella. Fragments of the TSPs can also be used according to the subject invention, particularly proteins comprising the phage receptor binding domains (PRBDs) which recognize their hosts and facilitate infection. The binding domains are specific to unique surface structures on bacteria and may be used for a variety of applications according to the subject invention. We have shown that by utilizing proteins comprising these PRBDs, it is possible to exploit the long-established evolutionary relationship between bacteria and their viruses (ie bacteriophages) that specifically infect them.

Such truncated proteins (a fragment of a TSP) can be referred to herein as phage receptor binding proteins (PRBP). Some PRBPs exemplified herein are a truncated version of a TSP, wherein the PRBP comprises the receptor binding/endorhamnosidase domain and the trimerization domain, without the head binding domain.

In some embodiments of the invention, there is provided a pharmaceutical composition comprising an effective amount of at least one PRBD formulated for delivery to the digestive tract of an animal in need of such treatment.

The subject invention also relates in part to novel, synthetic forms of truncated tail spike proteins comprising a PRBD. In some preferred embodiments, these are hexamers.

### BRIEF DESCRIPTION OF THE DRAWINGS

FIG. 1. P22sTsp and its Salmonella O-antigen receptor. The protein is shown with its Salmonella O-antigen receptor bound. On the right, the chemical structure of Salmonella O-antigenic repeating units is shown. Tsp shows a relaxed specificity in terms of the terminal sugar residue (Tyv, Abe or Par). The arrow is pointing at the cleavage site. The figure is based on FIGS. 1 and 2 of Steinbacher et al (Steinbacher, S. et al., 1996).

FIG. 2. Recombinant P22sTsp construct. (A) Schematic presentation of the P22sTsp construct depicting its two domains and added His₆ and RSGC sequences. (B) Sequence depictions of P22sTsp3 and P22sTsp5. The actual Tsp sequence is in bold. The mutated residues at positions 520, 561, 582, 584, 590 and 599 are shown (see Table 1 for more details).

FIG. 3. Schematic representation of various recombinant P22sTsps. Tsp figures were taken from Steinbacher et al (Steinbacher, S. et al., 1996).

FIG. 4. ELISA showing the binding of P22sTsp5 (hexamer) and P22sTsp5^{-x} (trimer) to Salmonella. (A) Scheme showing the assay format. (B) SEC showed that trimers and hexamers did not inter-convert over the course of the assays. In all cases, 50% binding occurs at 70 ng/mL.

FIG. 5. A. Overview of the two protocols used for animal studies. At time zero, chicks were inoculated with 10⁷ Salmonella. In Protocol 1, chicks were gavaged immediately after inoculation (1 h) with P22sTsp in 10% BSA or with 10% BSA alone. The next two gavages were given at 18 h and 42 h. In Protocol 2, the first gavage was delayed by 17 hours and given at 18 h. (B) Effect of orally administered P22sTsp5 on Salmonella colonization of chick ceca (i) and infection of liver and spleen (ii). Non-infected chicks had no Salmonella in their cecal contents (n=14). CFU=colony forming unit.

FIG. 6. Effect of orally administered P22sTsp5 on Salmonella colonization of ceca (A) and infection of liver (B) and spleen in chicks at an inoculation level of 10⁴ bacteria (second repeat). Protocol 1 was followed. The P22sTsp treatment group was done in duplicate as shown by numbers 1 and 2 (two different cages). Non-inoculated chicks had no bacteria detected in their ceca (n=9). In (B), two outliers for 10% BSA (5375) and P22sTsp/10% BSA-2 (10025) are not shown but taken into consideration for the calculation of medians. Medians are highlighted yellow on the graphs.

FIG. 7. Effect of orally administered P22sTsp on Salmonella colonization of ceca (A) and infection of liver (B) and spleen in chicks at an inoculation level of 10⁵ bacteria (third repeat). Protocol 1 was followed. The P22sTsp treatment group was done in duplicate as indicated by numbers 1 and 2 (two different cages). Non-inoculated chicks had no detectable bacteria in their ceca (n=9). In (B), one outlier for 10% BSA (3250) is not shown but taken into consideration for the calculation of medians. Medians are highlighted yellow on the graphs.

FIG. 8. P22sTsp reduces Salmonella motility. The dimensions of the Salmonella zone of motility on soft agar plates were observed and measured at different time points (A) and used to calculate motility areas. A graph of motility area versus incubation time was subsequently plotted (B).

FIG. 9 illustrates naturally occurring and novel configurations of P22 tail fibers.

FIG. 10 illustrates novel applications of tail spike proteins for food safety and disease control.

### BRIEF DESCRIPTION OF THE SEQUENCES

SEQ ID NO:1 is the DNA sequence encoding P22sTsp5H^{-X} (enzyme mutant, head-to-head hexamer configuration). For all the applicable sequences, the target of the mutant is indicated by underlining and light shading.

SEQ ID NO:2 is the amino acid sequence encoded by SEQ ID NO:1.

SEQ ID NO:3 is the DNA sequence of P22sTsp5^{-X} (enzyme mutant, tail-to-tail hexamer configuration).

SEQ ID NO:4 is the amino acid sequence encoded by SEQ ID NO:3. Note: when expressed in E. coli, the final product does not have the starting Met residue.

SEQ ID NO:5 is the DNA sequence of P22sTsp5H (wild-type, head-to-head hexamer configuration).

SEQ ID NO:6 is the amino acid sequence encoded by SEQ ID NO:5.

SEQ ID NO:7 is the DNA sequence of P22sTsp5 (wild-type, tail-to-tail hexamer configuration).

SEQ ID NO:8 is the amino acid sequence encoded by SEQ ID NO:7. Note: when expressed in E. coli, the final product does not have the starting Met residue. The seven) cysteine residues are indicated in bold, italics, larger font, and dark shading.

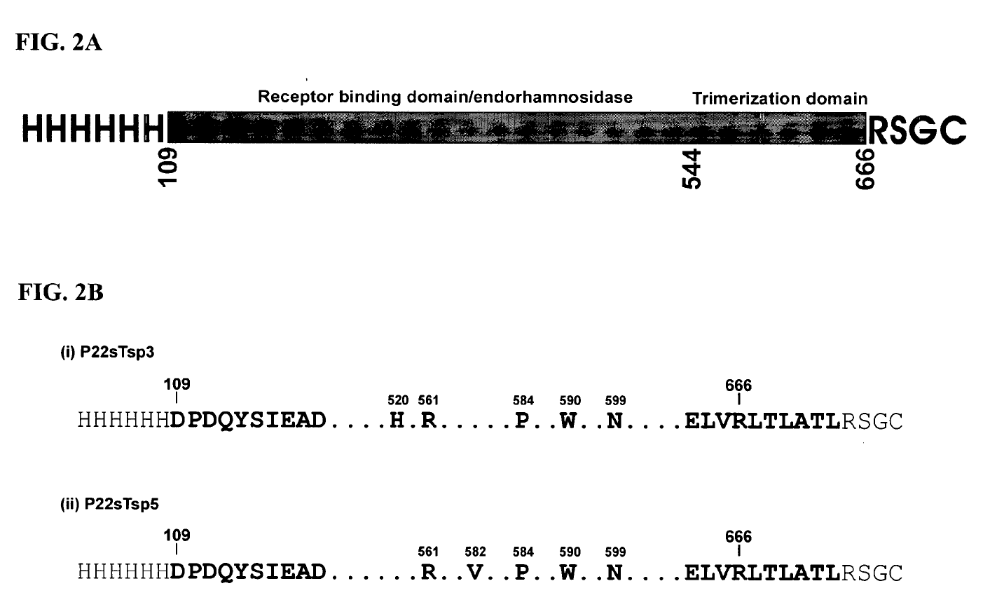

SEQ ID NO:9 is the amino acid sequence of a wild-type Tsp, having endorhamnosidase activity, from Enterobacteria phage P22 (protein accession No.: AAF75060). (Unless otherwise specified or determinable, the untruncated Tsp is the reference for numbering; see residues 108 versus 109 in FIG. 2, for example. In addition, some protein sequence numbering might not take into consideration the start codon, Met. Thus, numbering can vary slightly, but equivalent numbering is readily determinable by sequence alignments.)

SEQ ID NO:10 is the amino acid sequence of S. enterica serovar Typhimurium bacteriophage ST64T TSP (protein accession no. AAL15537).

### DETAILED DISCLOSURE OF THE INVENTION

The subject invention relates in part to novel uses of bacteriophage tail spike proteins (TSPs). Some preferred uses are therapeutic uses in animals, such as chickens, against pathogenic bacteria, such as Salmonella. Some tail spike proteins for use according to the subject invention naturally form trimers. Some TSPs for use according to the subject invention are naturally from the tail fibers of phages and are responsible for host recognition.

As used herein, reference to “isolated” polynucleotides, genes, and/or proteins, and/or “purified” proteins, refers to these molecules when they are not in environments, and/or not associated with other molecules, in which they would be found in nature. Thus, reference to “isolated” and/or “purified” signifies the involvement of the “hand of man” as described herein. For example, an “isolated” tail spike protein (TSP) of the subject invention signifies, for example, a TSP that is not in its naturally state and that is, for example, disassociated with a whole phage/phage capsid or head.

Fragments of the TSPs can also be used according to the subject invention, particularly proteins comprising a phage receptor binding domains (PRBD) which recognize their hosts and facilitate infection. Such proteins can be referred to as phage receptor binding proteins (PRBPs).

The binding domains are specific to unique surface structures on bacteria and may be used for a variety of applications according to the subject invention. We have shown that by utilizing PRBPs (proteins comprising a PRBD), it is possible to exploit the long-established evolutionary relationship between bacteria and their viruses (i.e. bacteriophages) that specifically infect them.

The subject invention also relates in part to novel, synthetic forms of tail spike proteins. In some preferred embodiments, these are hexamers.

The subject invention also includes polynucleotides that encode proteins of the subject invention, and polynucleotides that can be used in the production of proteins of the subject invention.

Trimers of the subject invention have three binding domains, while hexamers of the subject invention have additional (six) binding domains. A monomer has a single binding domain. We found that a single trimer, for example, is able to cross link bacteria as shown in our early aggregation experiments. In addition, in some embodiments, we have shown that a TSP retards the motility of Salmonella in motility assays.

The uses of these TSPs and/or PRBPs include direct use as non-antibiotic antimicrobials for the control of enteric pathogens in animal and human therapy, as well as for identification of novel antigens for the discovery and development of vaccines. Uses of PRBPs according to the subject invention include providing to/administration to animals, particularly (in some embodiments) to gastrointestinal tracts of animals (including humans) and to oral cavities (including the human mouth, to combat gingivitis, for example; such administration can be in the form of toothpaste and/or mouthwash comprising the PRBP(s)). The subject invention can also be formulated and administered to combat a variety of diseases and pathogens, such as Clostridium difficile (in the colon)/colitis. (The complete genome sequence of Clostridium difficile phage C2 is now known and has been compared to C. difficile phages CD 119 and CD630, and to Strep, pneumoniae phage EJ-1. Goh et al., Microbiology 153 (2007) 676-685.) There are also applications in the food industry (to target undesireable microbial growth in yogurt fermentation, for example, so the desired organisms can thrive).

As used herein, “providing,” “administering,” and or “treating” include any methods in which a protein of the subject invention can be used for a desired purpose of the subject invention. Such methods include injection, making the protein available on feed, in sprayable formulations, in ingestible formulations and compositions, and the like. An “effective amount” is an amount of the protein that is suitable for achieving the desired end result. For example, chickens injected or fed/ingesting an effective amount of the protein will have protection from a pathogenic bacteria to the extent that losses caused by sickness are decreased. Formulations that achieve this purpose would comprise an effective amount of the active ingredient/protein of the subject invention. Sprayable formulations comprising an effective amount of a PRBP of the subject invention are suitable for delivering an effective amount of the PRBP to, for example, surfaces used in hospital or meat treatment facilities, so that a reduction of target organisms is achieved. Total sterilization is not required, though this level of cleanse can be obtained with some uses.

Other uses may also include the use of these PRBPs as diagnostic sensors for the detection and identification of specific bacteria of interest. Some possible diagnostic uses are discussed in more detail below.

The PRBPs are easily cloned and over-expressed in a variety of recombinant host systems. For example, in the case of the P22 bacteriophage that infects Salmonella, it forms a self assembling Tail Spike Protein (TSP) complex that may function as a recombinant protein scaffold for the production a various types of fusion proteins for a wide variety of applications.

Bacteriophages have already adapted to recognize specific structures that are exposed and common to a particular organism and these structures provide novel bacterial targets for therapeutic and diagnostic purposes. Bacteriophages are ubiquitous, abundant, highly specific viruses that can be used to identify unexpected and novel surface exposed bacterial targets that can be used as diagnostics or in therapy development against any bacterial pathogen.

Phage receptor binding domains (PRBDs) determine bacteriophage specificities for their bacterial hosts. Bacteriophages are diverse in specificity and thus provide a myriad of PRBDs, each specific for a particular bacterial pathogen. The host specificity and the genome sequence of many phages, including those specific to pathogens of high public concern, are already known. Thus in many cases, acquiring a bacterial pathogen-specific PRBD may involve mainly a cloning step. In some embodiments, these PRBDs may be used for prevention of food pathogens at source by oral administration. In alternative embodiments, there is provided the use of PRBDs in a scaffold for surmounting pathogen-specific binding domains for use in prevention at source applications.

In some aspects of the invention, there is provided a PRBP comprising at least one phage receptor binding domain having binding affinity for at least one bacterial ligand including a protein or sugar. As discussed herein, at least one PRBP/PRBD may be arranged to be administered orally. Formulations that are known in the art can be used according to the subject invention to allow delivery of the PRBD to a specific region of the digestive tract, for example. Accordingly, in some embodiments, there is provided a pharmaceutical composition comprising an effective amount of at least one PRBP/PRBD formulated for delivery to the digestive tract of an animal in need of such treatment.

As used herein, an animal in need of such treatment' is not necessarily limited to animals suffering from a disease or disease-like symptoms due to a bacterial infection but also includes for example, an animal intended for slaughter. This includes, for example, livestock, poultry, or the like to which at least one PRBD or a mixture of PRBDs for common bacterial pathogens (either specific to the animal or common to the geographic region) is administered.

Compositions of the subject invention can be administered to/used to treat various animals including humans and “production animals” including cows, chickens (egglayers and for meat), pigs, fish, and other livestock in general.

A ‘bacterial pathogen’ is not necessarily pathogenic to the animal itself but may be pathogenic to an animal that will come in contact with the products derived from the animal for slaughter. In yet other embodiments, the pharmaceutical composition may include PRBDs directed against one or more bacterial pathogens as described above that are shed by the animal ‘in need of such treatment’, thereby preventing bacterial contamination of other animals or of the local environment, such as irrigation water and surface water (runoff). Examples include but are by no means limited to shedding of bacterial pathogens by cattle or swine in a pen such that piglets or calves are infected by the bacterial pathogens.

In other embodiments, the animal in need of such treatment may have or may be suspected of having a bacterial infection and the pharmaceutical composition may comprise at least one PRBP/PRBD directed against the suspected bacterial pathogen or may comprise PRBDs directed against a number of common pathogens.

According to the subject invention, a variety of pathogens can be targeted, and a variety of hosts can be treated. A variety of TSPs/PRBPs/PRBDs can accordingly be selected for use according to the subject invention. Some hosts and PRBPs are mentioned elsewhere. Further examples include the pathogen E. coli 0157 in cattle. Contaminated runoff from was implicated in pathogenic E. coli contamination of adjacent spinach fields, as well as apple orchards (and cider prepared therefrom). More specifically, there was the 2006 North American E. coli outbreak involving foodborne E. coli 0157:147, a potentially deadly bacterium that can cause bloody diarrhea and dehydration. The initial outbreak occurred in September 2006 and involved fresh spinach. A subsequent outbreak, in November-December 2006, was initially attributed to green onions but later believed to have been caused by prepackaged iceberg lettuce. The initial outbreak was traced to organic fresh spinach grown on a small farm in California. Investigators with the Center for Disease Control speculated that the dangerous strain of bacteria originated from irrigation water contaminated with cattle feces. See Wikipedia.org.

Earlier, in 2000, the small community of Walkerton, Ontario, Canada was struck by a waterborne disease resulting from cattle manure washing into a shallow water supply well. Lab results confirmed the presence of Campylobacter bacteria and E. coli 0151:H7. DNA testing identified the contaminating source as a cattle farm a short distance away from the well. Experts confirmed that heavy rainfall carried manure from the cattle farm close enough to permeate and corrupt the water source. See website: waterandhealth.org/drinkingwater/fiveyears.html.

Prophylactic uses are included within the scope of the subject invention. PRBPs of the subject invention can be incorporated into feed and/or water for animals, for example.

Humans can also benefit from the subject invention. For example, pathogens associated with gingivitis can be targeted with a variety of formulations according to the subject invention, including toothpaste and mouthwash. PRBPs of the subject invention can be included in deodorants, for example. Additional details regarding formulations and other appropriate uses are discussed in more detail below and elsewhere herein.

In some embodiments of the invention described herein, more than one PRBD for a given bacterial target may be used. These may be from closely related phages or may be from less closely related phages. As will be appreciated by one of skill in the art, the use of multiple PRBDs for a single bacteria may in some embodiments greatly increase effectiveness. In addition, compositions of the subject invention can optionally comprise additional active ingredients to target multiple pathogens (for example), and/or to target the same pathogen (for example) by using an additional mechanism of action. For example, antibodies (against Salmonella, for example), antibiotics, and small molecular weight compounds can also be included in some formulations.

In other embodiments, there is provided at least one PRBP/PRBD directed against a bacterium of interest mounted to a support. In some embodiments, the PRBDs bound to the support may be used as part of a detection device for detecting bacteria in a flowable fluid, for example, air or a liquid such as water. That is, the phage receptor binding domain is used advantageously to bind to bacteria that are flowed over the support, thereby allowing the levels of pathogens of interest within the flowable fluid to be detected. Such embodiments can include uses for determining baciliform/fecal coliform counts in drinking water or lake water, for example.

In other embodiments, the PRBDs mounted to a support may be arranged for removal of the pathogens from the flowable fluid. As will be appreciated by one of skill in the art, in many cases, this represents a question of scale of the support and/or density of PRBDs mounted thereto. Such embodiments could include uses in water filtration devices, such as those for home use. Certain pathogens, such as those that are relatively more prevalent or common, could be targeted for removal/purification with water filters in this fashion.

In yet other embodiments, the PRBDs may be applied as one would apply a disinfectant, for example, as a powder or fluid, to surfaces at risk of or suspected of bacterial contamination. As discussed herein, while not wishing to be bound to a particular theory, it is of note that many PRBDs bind to bacterial flagella and/or to bacterial cell surface proteins. Given that bacterial motility and/or cell surface proteins are often required for infection or retention of bacteria, blocking these cell surface binding sites and/or reducing motility of the bacteria will reduce the infectivity of the bacteria. An Example regarding motility inhibition is provided below.

The subject PRBPs can also be used in a variety of industrial biocidal applications. They can be used for the preservation of latex, and paints in cans, for example. They can be used in cosmetics and for the preparation thereof. They can be used as hard surface disinfectants for hospitals, medical devices, catheters, and the like. Biofilms, in particular, can be targeted in a wide variety of situations. Cold sterilization for biomedical uses, where autoclaves are not suited, are also excellent applications that are now enabled by the subject technology. Stents and the like can be treated according to the subject invention. Water can also be wholly or partially disinfected according to the subject invention. Carcasses can also be treated to wholly or partially “sterilize” them, for example. Industrial (and residential) uses for such applications include cooling water, heat exchanges (in air conditioning uses and the like), and the like. Such apparatus can be found in a variety of situations, such as in the pulp and paper industry.

A variety of additional uses are also now possible. For example, such uses include water filters (PRBPs mounted thereon), clean up of surfaces that have been contaminated or potentially contaminated by bioterrorist attacks or by other similar and/or unintentional contamination (mail/postal, offices, air ducts, and the like).

Legionella, for example, can also be targeted according to the subject invention, in a variety of situations. This can be targeted in hospital environments, in water supplies, and in cooling towers, air ducts, and the like.

A variety of formulations can be made for the particular end uses, as would be known by one skilled in the art having the benefit of the subject application. Various carriers are known in the art and could be adapted for use according to the subject invention. Various solvents, for example, could be used in delivery formulations, including water-based formulations.

A common denominator, so to speak, of various applications according to the subject application would be inactivation and/or prevention of colonization by a pathogenic (or other target) bacteria. The subject invention can be used with practically any bacteria that is infected by a phage wherein the phage has a TSP that binds somewhere on the surface of the bacteria. Uses can be but do not need to be bio“eidal”; they can also cause bio“static” end results.

As will be appreciated by one of skill in the art, PRBDs may be identified by a variety of means known in the art. As discussed above, PRBDs for many bacteriophage are already known or can be identified based on sequence homology or gene location within the phage genome. Such peptides are often referred to as either docking or attachment proteins or may be fiber, spike or tail proteins. However, uses of these phage receptor binding proteins (PREP) according to the subject invention are novel.

As will be appreciated by one skilled in the art, the use of PRBDs instead of the whole phage may reduce the problem of emergence of resistant hosts associated with phage therapy. Furthermore, due to their natural multimericity and stability, PRBPs/PRBDs do not require avidity and stability engineering to render them efficacious for prevention-at-source applications.

A well-known recombinant protein from a trimeric tail spike protein (TSP) of the P22 bacteriophage can be used according to the subject invention to act as a protein scaffold for engineering various recombinant proteins of interest. Each tail fiber in Salmonella phage P22 naturally exists as a trimer. We also engineered novel, hexameric forms of these proteins. These can assemble in various novel configurations, including head-to-head, head-to-tail, and tail-to-tail configurations, as illustrated.

Sequences exemplified herein in the 560 amino acid range contain sugar binding domains and domains that are responsible for trimerization. The head or capsid binding domain of the TSP was omitted in these embodiments.

Exemplified proteins have been engineered to form a hexamer by adding amino acids on either end of the protein to form a recombinant protein. The resulting structure forms di-sulfide bonds between the terminal ends of the protein and thus forms a homo hexameric structure and has been shown to bind to Salmonella, its natural host, and agglutinate cells. This novel composition has been used as a high affinity binding protein to reduce the colonization of Salmonella in the gut of animals such as chicken. Due to its specificity to Salmonella, this protein is also a diagnostic protein for the detection of Salmonella.

In addition to uses of these proteins as a therapeutic protein, due to the self-forming trimeric and hexameric structure it may also be useful as a recombinant scaffolding protein to attach other recombinant proteins of interest. Examples include any high affinity binding proteins including but not limited to single domain antibody fragments. In addition to binding proteins, this scaffold could be used as a fusion protein for other recombinant proteins of interest such as antigenic proteins to be presented as antigens.

Due to the non-glycosylated nature of the TSP scaffold, it may be expressed on various expression systems including prokaryotic and eukaryotic systems (such as Pseudomonas fluorescens, yeast, and plants) TSPs have been expressed in E. coli cells and were purified by conventional protein purification procedures. This TSP will agglutinate Salmonella cells at 4° C. following overnight incubation. In addition, TSP dosed by oral gavage at a dose of 33 micrograms per dose reduced the colonization of Salmonella in chickens 400 fold as compared to controls treated only with BSA as a control. See also the motility inhibition Example below.

The use of bacteriophage proteins according to the subject invention offers several advantages. For example, because of their specificity, they will not disrupt host flora. They are nontoxic and are regularly consumed in foods (usually more than 10⁺⁸ phages per gram of meat). Phages are only composed of proteins and nucleic acids, so there are no harmful breakdown products. Phages are especially abundant in the gastrointestinal tract. Oral toxicity studies have shown no adverse effects in rats repeatedly dosed with 10⁺¹¹ phages. Phages have recently been approved by the FDA for use on meat and poultry products. For example, the FDA has recently approved the first use of intact bacteriophage cocktails to be added to ready-to-eat meats and poultry products to protect consumers from L. monocytogenes. L H Lang, “FDA approves use of bacteriophages to be added to meat and poultry products”. Gastroenterology 131 (2006).

Phage components are relatively cheap to produce (they can be considered to be medicine that multiplies). They can also readily be expressed at therapeutic scales.

In addition, they offer rapid activity within minutes and a high rate of success. Any resistance that develops will render bacteria less virulent because the phage target key surface structures. Phages also continue to evolve along with bacteria, thus offering limitless generations of new therapies to tap into. Phage can be effective against multidrug-resistant bacteria.

For additional information regarding the use of TSPs and PRBPs derived from phage of Campylobacter jejuni, particularly φ1 and φ2. See U.S. Ser. No. 60/909,044 entitled “Interactions between Campylobacter jejuni and bacteriophages,” filed Mar. 30, 2007. Such phage are in the Myoviridae family of the Order Caudovirales.

Recently, phages were isolated that exhibited differential lytic activities to various C. jejuni strains examined for viral infection from the Russian Federation. Two bacteriophages had contractile tails considered morphotype A1 of the family Myoviridae while a third had a long non-contractile tail of morphotype B1 in the family Siphoviridae. A fourth phage had an icosahedral head that was classified as morphotype B1 of the Siphoviridae, while a fifth phage had an icosahedral head with a short tail of morphotype C1 in the Podoviridae. Transmission electron micrographs of a few representative Campylobacter phages were obtained. See the Chapter entitled, “Bacteriophage Therapy and Compylobacter,”of Campylobacter, 3rd Edition by ASM Press; editors Nachamkin, Szymanski and Blaser; available for sale June 2008.

Still further, there is diversity of phage in nature. Knowledge about phage biology, genomics, isolation, and the like has accumulated. Phages have evolved to withstand harsh environmental conditions.

Some preferred phages include those of the Order Caudovirales. This Order includes phage of the Family Siphoviridae. This Family includes many phage of enteric bacteria, and other phage that are of interst. For a more specific discussion and list, see website ncbi.nlm.nih.gov/ICTVdb/Ictv/fs_sipho.htm.

Also of interest are phage from the Order Caudovirales that are in the Family Myoviridae. Various phages in this Order and Family, especially those having TSPs having structural features like those of other TSPs discussed or suggested herein, can be used according to the subject invention. For example, bacteriophage Det7 is a phage of Salmonella enterica. This phage is of the Family Myoviridae but its TSP is like that of a Podoviridae. Thus, Podoviridae-like TSPs can be used according to the subject invention, even if the TSPs are from whole phage of a different Family, for example, so long as the TSPs are structurally similar. The three-dimensional structure for this protein, and for other phage proteins for use according to the subject invention, have been solved. See e.g. Walter et al., J. Virology, Vol. 82, No. 5, Mar. 2008, pp. 2265-2273.

In addition, a podoviral-like TSP has also been found to be specific to Shigella. This protein has been found to be a structural homolog of the P22 TSP but without a high degree of sequence similarity in the receptor binding domain. Freiberg et al., “The Tailspike Protein of Shigella Phage Sf6,” J. Biol. Chemistry, Vol. 278, No. 3, Jan. 2003, pp. 1542-1548.

As mentioned above and elsewhere herein, one preferred Family of the Order Caudovirales is the Family Podoviridae, which includes Enterobacteria phage T7, Bacillus phage φ29, Enterobacteria phage P22, and Enterobacteria phage N4. Phage in this Family tend to have TSPs that are structurally similar (even if their amino acid sequences are divergent).

Exemplary Podovirdae for use according to the subject invention include the following, where a “*” denotes pathogenic bacteria/Podoviridae pairs.

| Genus/species of the host strain | Phage | Bacterial host |
|---|---|---|
| Acinetobacter calcoaceticus | | 531 |
| | | A10 |
| | | A3 |
| | A3/2 | 2 |
| | A10/A45 | A45 |
| | B9PP | Mx70.71 |
| | A36 | A28 |
| | E13 | 75.53 |
| | E14 | 75.126 |
| | B9GP | MX70.71 |
| | BS46 | AC 45 |
| Acinetobacter calcoaceticus | 531 | 9956 |
| var. anitratus | | |
| Acinetobacter genotype 16 | 205 | ATCC 17988 |
| Acinetobacter haemolyticus | 2213/73 | 2213 |
| Acinetobacter johnsonii | 133 | N/A |
| Actinomyces viscosus | Av-1 | MG-1 |
| Aeromonas hydrophila | Aeh1, T7-Ah | C-1 |
| | Aeh2 | ATCC 7966 |
| | PM4 | AH-44 |
| Aeromonas salmonicida | 3, 31 Popoff, 56 Popoff | 95-68 |
| | 25 | 170-68 |
| | 44RR2.8t, 59.1, Asp37 | 866 |
| | 32 Popoff | 132-66 |
| | 43 Popoff | 01-J3000 |
| | 29 Popoff, 51 Popoff | Oct-69 |
| | 65 Popoff | 35-69 |
| Aeromonas veronii (ssp. biovar | PM6 | AH-42 |
| sobria) | | |
| Anabaena variabilis | A-1-L | EPA#261 |
| Aneurinibacillus | | KAS232 |
| aneurinilyticus | | |
| | ΦBA-1 | KA23 |
| Arthrobacter globiformis | AGL4 | CCM 1650 |
| Asticcacaulis biprosthecium | bla | Dev13 |
| | ΦAcS3, ΦAcS4 | C-19 |
| Bacillus anthracis | | Sterne CN 35-18 |
| | γ, AP50 | CN 18-74 |
| Bacillus cereus | CP-54Ber, TP-15c | 13472 |
| | Bace-11 | S154-2 |
| Bacillus clarkii | BCJA1c | JAD |
| Bacillus licheniformis | GA-1 | G1R |
| | θ1, θc, LP52 | UM-12 |
| | | UM-12/qb/Def |
| Bacillus megaterium | αc3 | tiberius |
| | MP13, MP15 | QM B1551 |
| | MJ-1, MJ-4 | F4 |
| | G | PGH |
| Bacillus pumilus | PBP1 | 706S |
| Bacillus sphaericus | 1A | SSII-1 |
| | SST | Kellen Q |
| Bacillus subtilis | Φ105, Φ3T, SP16 | W168 |
| | PBS1 | SB19E |
| | SP-15 | W23 Sr |
| | SP8 | Marburg |
| | SP10 | N/A |
| | SPP1 | SB168 (trp-) |
| | | CU1985 |
| | SP50 | W23 |
| | SPβ | CU1050 |
| | H1 | 1G20 |
| | Φ15, Φ29 | 110NA |
| | | CU 7004 |
| | | IL8 |
| | | IL1 |
| | F, SN45 | ATCC 27505 |
| | H2 | CU 3431 |
| | BS5 | ATCC 15841 |
| | S-a | 15841 |
| | CS1 | F6 |
| | SPO1 | CB10 |
| Bacillus thuringiensis | Bastille | N/A |
| | P400 | 2 |
| | Bat1 | 1 |
| | Bat5, PK1 | 6 |
| | Bat7 | 3 |
| | Bat10, Bat11 | 23 |
| | Bat18 | 18 |
| | B.1715V1 | berliner 1715Wt |
| | Tb10 | Berliner T06A001 |
| | Tg4 | MUL-B 04: 1.1 |
| | DP7 | As-IV-5B3 var. galleria 087 |
| | Bam35 | T24 |
| | GP-10 | HD-73 |
| | | B16 |
| Bacillus thuringiensis (ssp. sv. | GIL01, GIL16 | GBJ002 |
| israelensis) | | |
| | | AND508 |
| Bacillus thuringiensis (ssp. var. | mor 1 | 505b |
| galleriae) | | |
| Bacteroides fragilis | Bfl | f28 |
| Bordetella avium | ΦATCC | 197N |
| Bordetella bronchiseptica | | 8101 |
| | | AGI-L |
| Bordetella parapertussis | 8101, Tohama | 504 |
| | | Tohama |
| | L-1 | 17903 |
| Brochothrix thermosphacta | BL3, MT | L90 |
| | A9, NF5 | NF4 |
| | | B12 |
| Brucella abortus | Fz, S708, Tb | 19 |
| (ssp. biotype1) * | | |
| Brucella canis * | R/C | Mex.51 (79/85) |
| Brucella melitensis | Bk | Isfahan |
| (ssp. biotype1) * | | |
| Brucella suis (ssp. biotype1) * | Wb | 1330 |
| Burkholderia cepacia * | 83-24 | 83-190 |
| | 42 | N/A |
| Caulobacter crescentus | ΦCbK, ΦCR30 | CB15 |
| Clavibacter michiganense | CN11, CN77, CN8, | CN18-5 |
| (ssp. nebraskense) | CNRH, CNX | |
| Clostridium perfringens | Φ3626 | WS2895 |
| Clostridium | HM2, HM3 | NI-4 |
| saccharoperbutylacetonicum * | | |
| | HM7 | NI-504 |
| Corynebacterium crenatum | B277 | N/A |
| Corynebacterium diphtheriae | | C7 (ω) 5tox |
| | ω | C7- |
| | β | C7 |
| Corynebacterium glutamicum | Cog | LP-6 |
| | CL31 | CL31 |
| Delftia acidovorans | ΦW-14 | N/A |
| Enterobacter cloacae | l | 73-833 (77) |
| Enterococcus faecalis | VD13 | 8413 |
| | 182 | 8384a |
| | VD1884 | D 3854 |
| | 1 | 8413 |
| Erwinia amylovora | PEa 31-2 | 110R |
| Erwinia herbicola | Erh1 | N/A |
| Escherichia coli * | Esc-7-11 | O44: K74 MUL-B37.2 |
| | E920g, pt1, T1, T2, T3, | B |
| | T4, T5, T6, T7 | |
| | | K12 C600 (λ) |
| | ΦX174 | C |
| | λvir | K12S |
| | RB69 | K12 (λ) Lederberg |
| | N4 | W3350 |
| | 121Q | MUL-B70.1 |
| | β4Q | O86: B7 MUL-B3.1 |
| | HK243 | K12 65 |
| | λ | K12S Lederberg |
| | BW-1 | K1 |
| | Haiti | N/A |
| | I2-2 | JE-1 (N3) |
| | PR64FS | JE-2(R62Rpilc) |
| | M | J53(RIP69) |
| | J | K12 J62-1(R997) |
| | PR772 | K12 J53-1(R15) |
| | C-1 | JE-1 (RA1::TN5Sqr) |
| | Φ92, pilHα | J62-1 (R27::TN7) |
| | D108, Mu | 40 |
| | | HM 8305 |
| | 1 | O157: H7 C-8299-83 |
| | 2 | O157: H7 E318 |
| | 3 | O157: H7 A7793-B1 |
| | 4 | O157: H7 C-8300-83 |
| | 5 | O157: H7 C-7685-84 |
| | 6 | O157: H7 CL40 |
| | 7 | O157: H7 C-7111-85 |
| | 8 | O157: H7 B1190-1 |
| | 9 * | O157: H7 B1328-C10 * |
| | 10 * | O157: H7 A8188-B3 * |
| | 11 | O157: H7 C7420-85 |
| | 12 | O157: H7 3283 |
| | 13 | O157: H7 C-7140-85 |
| | 14 | O157: H7 5896 |
| | 15 | O157: H7 C-7142-85 |
| | 16 | O157: H7 C-91-84 |
| | | K12 C600 (H-19J) |
| | H-19J, P1kc | K12 C600 |
| | R17 | CSH39 |
| | | K12 C600 (933-J) |
| | Ω8 | F492 (O8: K27-: H-) |
| | O103 | O103 2929 |
| | K20, SS4 | K12 MC4100 |
| | K30 | E69 O9: K30: H12 |
| | O9-1 | CWG 1028 |
| | HK97 | Ymel mel-1 supF58 |
| | | Ymel (HK97) |
| | | 0103 GVs |
| | P1D | Rougier |
| | TC4 | TC4 |
| | MB4 | MB4 |
| Flavobacterium johnsoniae | | UW10123 |
| | ΦCjT23 | UW10136 |
| | ΦCj1, ΦCj27, ΦCj7 | UW101 |
| Gluconobacter | Werquin | N/A |
| Gordonia rubripertincta | NJL | CF222 |
| Haemophilus | ΦAa17 | ATCC 29524 |
| actinomycetemcomitans | | |
| Haemophilus influenzae | | Rd-L-10 |
| | HP1 | Rd-001 |
| Hafnia alvei | 1672 | 1672 |
| Halobacterium salinarum | Ja.1, S45 | NRC 34001 |
| | ΦH, ΦN | NRL R1 |
| Janthinobacterium | JX1 | T-5 |
| halosensibilis | | |
| Klebsiella aerogenes | FC3-9 | C3 |
| Klebsiella pneumoniae | K13 | G162RIP69 substrain B |
| Kluyvera cryocrescens | Kvp1 | 21g |
| Kurthia zopfii | 3/K26 | 26 |
| | 6/K27 | 27 |
| Lactobacillus paracasei | PL-1 | ATCC 27092 |
| (ssp. paracasei) | | |
| Lactobacillus plantarum | fri | A |
| Lactococcus lactis | P001 | P001 |
| | 936 | 158 |
| | 949 | ML8 |
| | 1358 | 582 |
| | P008, P270, P335, P369 | F7/2A |
| | KSY1 | IE-16 |
| | c6A | C6 |
| | 1483 | 111 |
| | PO87 | C10 |
| | P107 | F7/2A |
| | | BK5 |
| | BK5-T | H2 |
| | c2, p2, sk1 | MG1363 |
| | | LM0230 |
| | ul36 | SMQ-86 |
| | bIL170, bIL67 | IL1403 |
| | | R1 |
| | Q54 | SMQ-562 |
| | 1706 | SMQ-450 |
| | | F7/2 |
| | Tuc2009 | UC509.9 |
| | rlt | R1K10 |
| Leuconostoc mesenteroides | pro2 | pro 2 |
| Leuconostoc mesenteroides | Φ400 | P1 |
| (ssp. cremoris) | | |
| Listeria innocua | | 4211 |
| | 4211 | 5290 |
| Listeria monocytogenes | 2685 | 1803 serovar l/2c |
| | 2671 | 10401 |
| | 2389 | PS 1089 |
| | H387 | IP31 |
| | ΦLMUP35 | WSLC1001 |
| | A511 | WSLC1003 |
| Marine bacterium | H7/2 | H7 |
| | H100/1 | H100 |
| | H106/1 | H106 |
| | 11-68C | Nov-68 |
| | H105/1 | H105 |
| Mesorhizobium loti | Φ2037/1 | NZP2037 |
| Micrococcus luteus | N1, N5 | 4698 |
| Mycobacterium | 33D (Warsaw), BK1, | N/A |
| | Bo4, Clark, D29, | |
| | DNAIII, L5, Legendre, | |
| | Leo, Roy, Sedge, | |
| | Wiseman | |
| Mycobacterium smegmatis | Baits, I3 | SN2 |
| | | mc2155 (L5ts43) |
| Oenococcus oeni | Lco22 | EFA 49 |
| Paenibacillus larvae | | 3558 |
| | PBLO.5c | 2605 |
| Paenibacillus polymyxa | IPy-1, SPy-2, Spy-3 | L |
| Pasteurella multocida | 32 | SHD |
| Proteus mirabilis * | 13/3a | 13 |
| Pseudoalteromonas espejiana | PM2 | BAL 31 |
| Pseudomonas aeruginosa * | 7 Lindberg | 72-235 |
| | 16 Lindberg | 76-89 |
| | 24 Lindberg, F7 | 72-238 |
| | Lindberg | |
| | 31 Lindberg | 72-239 |
| | 44 Lindberg | 81-262 |
| | 68 Lindberg | 72-241 |
| | 73 Lindberg | 76-51 |
| | | 72-238 |
| | F8 Lindberg, SD1-M | 72-115 |
| | F10 Lindberg | 72-19 |
| | 109 Lindberg | 72-237 |
| | 119x Lindberg * | 76-52 * |
| | 352 Lindberg | 76-116 |
| | 1214 Lindberg | 72-249 |
| | M4 Lindberg | 72-250 |
| | M6 Lindberg | 76-73 |
| | 2 Lindberg, D3, E79, | PAO1 * |
| | F116L * | |
| | D3112, PB-1 | N/A |
| | ΦKZ, 21 Lindberg | PAO1 |
| | ΦPLS27 * | AK 44 * |
| | | AK 1012 |
| | KF1 | PML28 |
| | | NIH S |
| | | PML14 (PS17)+ |
| | PS17 | PML14 |
| | PP7 | PA01 |
| | Pfl | K |
| Pseudomonas fluorescens | P10 | P10 |
| Pseudomonas putida | gh-1 | ATCC 12633 |
| | LU11 | LU11 |
| Pseudomonas syringae var. | Φ6 | HB1OY #3 |
| phaseolicola | | |
| | Φ13, Φ8 | LM2849 |
| Rhizobium | Φgal-1/ow, Φgal-1/R | Gal-1 |
| | Φgal-3/ow, Φgal-3/R | Gal-3 |
| Rhodobacter sphaeroides | RS1 | 2.4, 1 |
| | ΦRsG1 | Y |
| Rhodococcus canicruria | ΦEC | N/A |
| Saccharopolyspora erythraea | 121 | ATCC 11635 |
| Saccharopolyspora rectivirgula | ΦFR114 | DSM 43 747 |
| Salinivibrio costicola | G3 | G3 |
| | UTAK | B1 |
| Salmonella anatum * | ε15 | N/A |
| Salmonella bareilly | Sab2 | 9368 |
| Salmonella choleraesuis | χ | LT2 SL688 |
| (ssp. choleraesuis ser. | | |
| typhimurium) | | |
| Salmonella heidelberg * | 1 heidelberg | #1 |
| | 2 heidelberg | #2 |
| | 3 heidelberg | #3 |
| | 4 heidelberg | #4 |
| | 5 heidelberg | #5 |
| | 6 heidelberg | #6 |
| | 7 heidelberg | #7 |
| | 8 heidelberg | #8 |
| | 9 heidelberg * | #9 * |
| | 10 heidelberg | #10 |
| | 11 heidelberg * | #11 * |
| Salmonella newport | 16-19, 7-11, 9266Q | C487 |
| | 2.5A | C259 |
| Salmonella paratyphi | Jersey | B type1 |
| | Beccles | B |
| Salmonella senftenberg | SasL4 | S-219/89 |
| | Sas L6 | S403/88 |
| Salmonella typhi | O1, ViI, ViII | ViA subtypeTananarive |
| Salmonella typhimurium | P22, PRD1 | LT2 (pLM2) 1217 |
| Serratia marcescens | 290F | 2170 |
| | | HY |
| | | HY (Y, U)- |
| | Y, κ (kappa), Ψ (Psi) | HY (Ψ, Y)- ink-34 |
| | η (eta), π (pi), σ (sigma) | CV/rc3 |
| | 3M, K19Q | 2170 |
| Shigella dysenteriae | | SH (P2) |
| | P2 | a SH |
| Shigella sonnei | C16 | Y6R |
| | | N/A |
| Sinorhizobium meliloti | NM1 | M9S |
| | 1 Lesley, T1 Lesley | R1220 |
| | 7a Lesley | R2043 |
| | 27 Lesley | DMG-175 |
| | 43 Lesley | Rh-26 |
| | N2 Lesley | MBA-9 |
| | N3 Lesley | AN3 |
| | N4 Lesley | AN4 |
| | N9 Lesley | AN9 |
| | A3 Lesley | AP3 |
| | A8 Lesley | AP8 |
| | M3 Lesley | MB3 |
| | M4 Lesley | MB4 |
| | M5 Lesley | MB5 |
| | 70 Lesley | 102F70 |
| | Φ4 | M11S |
| | Φ10, CM1 | M12S |
| | MM1H | M14S |
| | ΦM11S | 444 |
| Sphingomonas paucimobilis | PAU | N/A |
| Staphylococcus aureus * | P68 | 68 |
| | 44AHJD * | 44A * |
| | 3A | 3A |
| | 77 | 77 |
| | 71 | 71 |
| | 187 | 187 |
| | 2638A | 2854 |
| | CS1, DW2 | DPC5246 |
| Staphylococcus carnosus | BaSTC2 | STC2 |
| Staphylococcus epidermidis | | 392 |
| | 392 | 414 |
| Staphylococcus hyicus | Twort | Twort |
| Staphylococcus saprophyticus | | 1154 |
| | | 1139 |
| | 1139 | 992 |
| | 1154A | 433 |
| Stenotrophomonas maltophilia | XMM1 | N/A |
| Streptococcus group C * | a/C7 | C7 Azgazavdah |
| Streptococcus mitis | | Hu-o8 |
| Streptococcus mutans | M102 | OMZ381 |
| Streptococcus pneumoniae * | Cp-1 | R6st |
| Streptococcus thermophilus | Ba 24 | 24 |
| | DT1 | SMQ-301 |
| | Q1 | SMQ119 |
| | 2972, 858 | RD534 |
| Streptomyces cattleya | CPC | N/A |
| Streptomyces chrysomallus | 17 | N/A |
| Streptomyces coelicolor | | A3(2) J1929 |
| Streptomyces levoris | SLE111 | IMET 41331 |
| Streptomyces lividans | ΦC31 | 1326 |
| Streptomyces venezuelae | MSP2 | S13 |
| Veillonella rodentium | N2 | N/A |
| Vibrio | α3A | 2 |
| | β, 16, 24, I, II, III, IV, | |
| Vibrio cholerae * | X29 | N/A |
| | Kappa | H218 Sm r |
| | 493 | O139 AJ27-493 |
| Vibrio cholerae (ssp. biotype El | 4996, 57, e4, e5 | Makassar 757 |
| Tor) * | | |
| | 13, 14, 32 | N/A |
| | | SLH 22 |
| | CP-T1 | 1621 |
| Vibrio natriegens | nt-1, nt-6 | N/A |
| Vibrio parahaemolyticus | VP1, VP11, VP12, VP6 | 3283-61 |
| | VP5 | K-40 pilot |
| | KVP20, KVP40 | RIMD2210001 (EB101) |
| | | VP33 |
| | Vf33 | VP19 |
| | Φ16, ΦHAWI-5, | 16 |
| | ΦPEL8C-1 | |
| Vibrio vulnificus | 71A-6 | MO6-24 |
| Xanthomonas campestris | HXX | SC114 |
| Xanthomonas oryzae | XP12 | (thy H) |
| Yersinia enterocolitica * | 2/F2852-76 | F2852-76 |
| | 4/C1334-76 * | C1334-76 * |
| | 3/M64-76, 5/C394-76 * | C394-76 * |
| | 6/C753-75, 7/F783-76 * | F783-76 * |
| | 8/C239-76 | C239-76 |
| | ΦYeO3-12 | 6471/76-c |
| Yersinia ruckeri | A41 | RS41 |

A variety of sources of TSPs and fragments thereof can be used. For example, other TSPs having endorhamnosidase activity can be used as a source, but the endorhamnosidase activity is not required in variants proteins/polypeptides derived therefrom for use according to the subject invention.

The subject invention now also offers alternatives where whole phage were previously used. Because there was no prior knowledge or expectation that TSPs or fragments thereof would be stable enough, on their own, to be used for the subject purposes, these uses are surprising and unexpectedly advantageous. For example, PRBPs can now be used for oral administration of PREP-impregnated feed to ayu fish (Plecoglossus altivelis) for protection against infection with P. plecoglossicida. Podoviridae phages that can be used as the source of TSPs and/or fragments and/or variants thereof include PPpA-1, PPpA-2, PPpA-3, PPpA-4, PPpW-2, and PPpW-4. (See e.g. Table 2 of Park et al., (2000) Appl. Environ. Microbial. 66: 1416-1422.) Another example is that PRBPs of the subject invention can also be added to ready-to-eat meats and poultry products to protect consumers from L. monocytogenes. Bacteria aggregated according to the subject invention can prevent attachment to various surfaces.

In addition, the subject invention reduces or eliminates concern regarding virulence gene transfer or transfer of potentially virulent unidentified ORFs.

According to the subject invention, one can exploit both temperature and lytic phages for therapeutics. Methods of storage and administration are better known for proteins versus intact phages. For example, stable proteins may be stored in frozen form at low temperatures, at ambient temperatures in the form of dry powders, or in stabling solutions without loss of virulence as would be the case for infectious phage. Various formulations of the subject invention can also be used to provide longer shelf lives for sensitive products, for example. In addition, such proteins may effectively be stored as genes in the form of cloned sequences in hosts such as bacteria or fungi or as isolated vectors.

Phages offer extensive opportunities for engineering (altering host specificity, formation of multimers, and the like). Tail spike proteins are inherently stable in gastrointestinal tracts. Tail spike proteins with differing specificities can be fused together. They agglutinate rather than lyse bacteria, so no harmful bacterial products are released. In addition, clonal originality is maintained during production, as opposed to intact phages which are prone to mutations.

Other TSPs and fragments thereof comprising a PRBD can be used according to the subject invention, particularly those having a 3D structure that is similar to the P22 TSPs exemplified herein. The influenza virus haemagglutinin is one example. See Steinbacher et al., J. Mol. Bio. (1997) 267, 865-880. Phage of the Podoviridae family, including Shigella SF6, are also ideal candidates. Freiberg et al., in particular, note that the TSP of Shigella Phage Sf6 is a structurally similar homolog of the P22 TSP without sequence similarity in the β-helix domain. (“The Tailspike Protein of Shigells Phage Sf6,” J. Biol. Chem. (2003), Vol. 278, No. 3, pp. 1542-48.) The carbohydrate/lipopolysaccharide binding domains from various phages can be used.

Thus, the subject invention includes the following embodiments:
A novel composition comprising a Tail Spike Protein (TSP), a modified recombinant TSP, and/or a PRBP comprising a Phage Receptor Binding Domain (PRBD). The PRBDs can be from a bacteriophage having a TSP that forms a multimeric structure to form the tail fiber. The modification can be the result of the addition of at least one cysteine residue to the terminal end of the protein.

Also included is
a formulation for use according to the subject invention, said formulation comprising a wild-type TSP. The subject invention also includes a mutant form of TSPs that form a hexamer in which the mutant form does not show enzyme activity of the wild type. An exemplified mutant has an amino changed in the enzyme active site at amino acid 392 that is changed from aspartate to asparagine. Other mutants are possible. Such hexamers include:
   a head to head variant hexamer, and
   a tail to tail variant hexamer. Polymers of such hexamers can also be formed, including tail to head, tail to tail, and head to head polymers.

Chimeric structures can be constructed using various phage binding domains:
within and the Podoviridae virus family,
within and the Myoviridae virus family,
and between various virus families such as Podoviridae and Myoviridae.

Phage binding proteins of the subject invention can be constructed by being modified for specificity via in vitro evolution techniques including error prone PCR, domain swapping, and direct amino acid substitutions.

Thus, the subject invention also includes a modified phage binding protein with changes in the trisaccharide binding domain to expand or direct the specificity of the PRBP.

TSPs, PRBPs/PRBDs, and recombinant variants thereof of the subject application can also be used for novel applications including the following:
for the control of enteric bacteria in animals (including humans and production animals),
as a diagnostic for the identification of bacterial types,
as a sensor that may be the initial sensor of a biosensor,
as a targeted delivery protein for drugs,
as a tool for the identification of antigens for the discovery and development of vaccines, and
as a protein scaffold for chimeric and/or fusion recombinant protein production.
   The subject application can be practiced using various production processes, including expression in any heterologous system including bacteria, fungi, animal cells, algae, and plants.

Multimers of the subject invention may be assembled in vivo or in vitro. Scaffolds of the subject invention may be chemically modified to carry other drugs of interest. See WO 03/046560. TSPs for use according to the subject invention are typically highly stable proteins and have an ideal structure as a protein scaffold.

The subject invention also includes
the use of PRBPs to identify novel therapeutic determinants for vaccine discovery,
and the use of phage binding fragments to identify novel therapeutic determinants.

PRBPs of the subject invention can be administered to, for example, the epidermis and exposed mucosal surfaces, including ocular, oral, nasal, lung, and lower mucosal surfaces. The mucosal surfaces in animals and humans, especially the gastrointestinal (GI) and respiratory tracts, are major portals of entry and/or sites of diseases caused by bacterial, viral, and parasitic pathogens. Examples of these diseases include those caused by enteropathogenic Escherichia coli, Campylobacter sp., Salmonella sp., Listeria monocytogenes, Helicobacter pylori, Shigella sp., rotaviruses and calciviruses in the gastrointestinal (GI) tract, and Mycoplasma pneumoniae, influenza virus, Mycobacterium tuberculosis, Streptococcus pneumoniae, severe acute respiratory syndrome (SARS) virus and respiratory syncytial virus in the respiratory tract. The urogenital tract is also a site of mucosal invasion/disease (e.g., those caused by human immunodeficiency virus, Neisseria and Chlamydia). The mucosal surfaces, especially in the respiratory tract, are also the sites where allergens (for example dust mites, pollen etc.) cause hyper immune responses resulting in allergic airway diseases such as asthma. However, the main, current approaches are to administer vaccines and typical antibiotics parenterally or systemically (for example by subcutaneous, intramuscular, intraperitoneal routes). Although these vaccines elicit immunity in the systemic compartment (bone marrow, spleen and lymph nodes), they fail to elicit immunity in the functionally independent mucosal compartment. Thus, the subject invention offers a completely new solution to these problems.

Accordingly, PRBPs of the subject invention can be incorporated in wound dressings (in a BAND-AID, for example). As discussed above, PRBPs of the subject invention can also be used in toothpaste (as they are very stable), in deodorant and other personal care items, in creams and lotions as a preventative, to prevent acne. PRBPs of the subject invention can also be formulated for use as disinfectants at slaughter houses (to wash machines and tools). Thus, they can be used as biocides and biostats. PRBPs of the subject invention do not need to be lethal to the target pathogens, but they can be effective in certain applications if the simply prevent colonization.

Any of a variety of suitable formulations can be used according to the subject invention, as would be appropriate for the desired end use. Formulations can include any standard pharmaceutical diluents. A diluent is a diluting agent. Certain fluid formulations, without a diluent, would be too viscous or too dense to flow sufficiently from one point to the other. To improve the otherwise restricted movement, diluents can be added. This decreases the viscosity of the fluids and improves their ability to flow and/or to be circulated.

Other formulation agents can be used, such as mixtures with pectin and/or other gut-active proteins, for example.

There are many other uses included in the subject invention. Embodiments of the subject invention have applications in food industries, for example. Proteins of the subject invention can be designed to compete with phage of Lactobacillus strains, for example, to improve yogurt yields. Thus, proteins of the subject invention can also be designed not only against pathogenic bacteria but also to outcompete undesirable phage to protect (by competitive binding to surface proteins of) beneficial bacteria.

Characterization of Proteins and Genes of the Subject Invention. Proteins and genes for use according to the subject invention can be obtained, identified, and/or defined by using and/or in terms of their ability to bind an oligonucleotide probe, for example. These probes are detectable nucleotide sequences that can be detected by virtue of an appropriate label or may be made inherently fluorescent as described in International Application No. WO 93/16094. The probes (and the polynucleotides of the subject invention) may be DNA, RNA, or PNA, for example. In addition to adenine (A), cytosine (C), guanine (G), thymine (T), and uracil (U; for RNA molecules), synthetic probes (and polynucleotides) of the subject invention can also have inosine (a neutral base capable of pairing with all four bases; sometimes used in place of a mixture of all four bases in synthetic probes). Thus, where a synthetic, degenerate oligonucleotide is referred to herein, and “N” or “n” is used generically, “N” or “n” can be G, A, T, C, or inosine. Ambiguity codes as used herein are in accordance with standard IUPAC naming conventions as of the filing of the subject application (for example, R means A or G, Y means C or T, etc.).

As is well known in the art, if a probe molecule hybridizes with a nucleic acid sample, it can be reasonably assumed that the probe and sample have substantial homology/similarity/identity. Preferably, hybridization of the polynucleotide is first conducted followed by washes under conditions of low, moderate, and/or high stringency by techniques well-known in the art, as described in, for example, Keller, G. H., M. M. Manak (1987) DNA Probes, Stockton Press, New York, N.Y., pp. 169-170. For example, as stated therein, low stringency conditions can be achieved by first washing with 2×SSC (Standard Saline Citrate)/0.1% SDS (Sodium Dodecyl Sulfate) for 15 minutes at room temperature. Two washes are typically performed. Higher stringency can then be achieved by lowering the salt concentration and/or by raising the temperature. For example, the wash described above can be followed by two washings with 0.1×SSC/0.1% SDS for 15 minutes each at) room temperature followed by subsequent washes with 0.1×SSC/0.1% SDS for 30 minutes each at 55° C. These temperatures can be used with other hybridization and wash protocols set forth herein and as would be known to one skilled in the art (SSPE can be used as the salt instead of SSC, for example). The 2×SSC/0.1% SDS can be prepared by adding 50 ml of 20×SSC and 5 ml of 10% SDS to 445 ml of water. 20×SSC can be prepared by combining NaCl (175.3 g/0.150 M), sodium citrate (88.2 g/0.015 M), and water, adjusting pH to 7.0 with 10 N NaOH, then adjusting the volume to 1 liter 10% SDS can be prepared by dissolving 10 g of SDS in 50 ml of autoclaved water, then diluting to 100 ml.

Detection of the probe provides a means for determining in a known manner whether hybridization has been maintained. Such a probe analysis provides a rapid method for identifying genes of the subject invention. The nucleotide segments which are used as probes according to the invention can be synthesized using a DNA synthesizer and standard procedures. These nucleotide sequences can also be used as PCR primers to amplify genes of the subject invention.

More specifically, hybridization of immobilized DNA on Southern blots with ³²P-labeled gene-specific probes can be performed by standard methods (see, e.g., Maniatis, T., E. F. Fritsch, J. Sambrook [1982] Molecular Cloning: A Laboratory Manual, Cold Spring Harbor Laboratory, Cold Spring Harbor, N.Y.). In general, hybridization and subsequent washes can be carried out under conditions that allowed for detection of target sequences. For double-stranded DNA gene probes, hybridization can be carried out overnight at 20-25° C. below the melting temperature (Tm) of the DNA hybrid in 6×SSPE, 5× Denhardt's solution, 0.1% SDS, 0.1 mg/ml denatured DNA. The melting temperature is described by the following formula (Beltz, G. A., K. A. Jacobs, T. H. Eickbush, P. T. Cherbas, and F. C. Kafatos [1982] Methods of Enzymology, R. Wu, L. Grossman and K. Moldave [eds.] Academic Press, New York 100:266-285):
1) Tm=81.5° C.+16.6 Log[Na+]+0.41(% G+C)−0.61(%formamide)−600/length of duplex in base pairs.
2) Washes are typically carried out as follows:
3) Twice at room temperature for 15 minutes in 1×SSPE, 0.1% SDS (low stringency wash).
4) Once at Tm-20° C. for 15 minutes in 0.2×SSPE, 0.1% SDS (moderate stringency wash).

For oligonucleotide probes, hybridization can be carried out overnight at 10-20° C. below the melting temperature (Tm) of the hybrid in 6×SSPE, 5× Denhardt's solution, 0.1% SDS, 0.1 mg/ml denatured DNA. Tm for oligonucleotide probes can be determined by the following formula: Tm (°C.)=2(number T/A base pairs)+4(number G/C base pairs) (Suggs, S. V., T. Miyake, E. H. Kawashime, M. J. Johnson, K. Itakura, and R. B. Wallace [1982] ICN-UCLA Symp. Dev. Biol. Using Purified Genes, D. D. Brown [ed.], Academic Press, New York, 23:683-693).

Washes can typically be carried out as follows:
1) Twice at room temperature for 15 minutes 1×SSPE, 0.1% SDS (low stringency wash).
2) Once at the hybridization temperature for 15 minutes in 1×SSPE, 0.1% SDS (moderate stringency wash).

In general, salt and/or temperature can be altered to change stringency. With a labeled DNA fragment >70 or so bases in length, the following conditions can be used:
Low: 1 or 2×SSPE, room temperature
Low: 1 or 2×SSPE, 42° C.
Moderate: 0.2× or 1×SSPE, 65° C.
High: 0.1×SSPE, 65° C.

Duplex formation and stability depend on substantial complementarity between the two strands of a hybrid, and, as noted above, a certain degree of mismatch can be tolerated. Therefore, the probe sequences of the subject invention include mutations (both single and multiple), deletions, insertions of the described sequences, and combinations thereof, wherein said mutations, insertions and deletions permit formation of stable hybrids with the target polynucleotide of interest. Mutations, insertions, and deletions can be produced in a given polynucleotide sequence in many ways, and these methods are known to an ordinarily skilled artisan. Other methods may become known in the future.

PCR technology. Polymerase Chain Reaction (PCR) is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki, Randall K., Stephen Scharf, Fred Faloona, Kary B. Mullis, Glenn T. Horn, Henry A. Erlich, Noiinan Amheim [1985] “Enzymatic Amplification of β-Globin Genomic Sequences and Restriction Site Analysis for Diagnosis of Sickle Cell Anemia,” Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. The extension product of each primer can serve as a template for the other primer, so each cycle essentially doubles the amount of DNA fragment produced in the previous cycle. This results in the exponential accumulation of the specific target fragment, up to several million-fold in a few hours. By using a thermostable DNA polymerase such as Taq polymerase, isolated from the thermophilic bacterium Thermus aquaticus, the amplification process can be completely automated. Other enzymes that can be used are known to those skilled in the art.

The DNA sequences of the subject invention can be used as primers for PCR amplification. In performing PCR amplification, a certain degree of mismatch can be tolerated between primer and template. Therefore, mutations, deletions, and insertions (especially additions of nucleotides to the 5′ end) of the exemplified primers fall within the scope of the subject invention. Mutations, insertions, and deletions can be produced in a given primer by methods known to an ordinarily skilled artisan.

Modification of genes and proteins. The genes and proteins useful according to the subject invention include not only the specifically exemplified full-length sequences, but also portions, segments and/or fragments (including internal and/or terminal deletions compared to the full-length molecules) of these sequences, variants, mutants, chimerics, and fusions thereof Proteins used in the subject invention can have substituted amino acids so long as they retain the characteristic binding/functional activity of the proteins specifically exemplified herein. “Variant” genes have nucleotide sequences that encode the same proteins or equivalent proteins having functionality equivalent to an exemplified protein. The terms “variant proteins” and “equivalent proteins” refer to proteins having the same or essentially the same biological/functional activity as the exemplified proteins. As used herein, reference to an “equivalent” sequence refers to sequences having amino acid substitutions, deletions, additions, or insertions that improve or do not adversely affect functionality. Fragments retaining functionality are also included in this definition. Fragments and other equivalents that retain the same or similar function, as a corresponding fragment of an exemplified protein are within the scope of the subject invention. Changes, such as amino acid substitutions or additions, can be made for a variety of purposes, such as increasing {or decreasing) protease stability of the protein (without materially/substantially decreasing the functionality of the protein). Variants wherein any changes are conservative changes, as discussed herein, are also included.

Variations of genes may be readily constructed using standard techniques for making point mutations, for example. In addition, U.S. Pat. No. 5,605,793, for example, describes methods for generating additional molecular diversity by using DNA reassembly after random fragmentation. Variant genes can be used to produce variant proteins; recombinant hosts can be used to produce the variant proteins. Using these “gene shuffling” techniques, equivalent genes and proteins can be constructed that comprise any 5, 10, or 20 contiguous residues (amino acid or nucleotide) of any sequence exemplified herein.

Fragments of full-length genes can be made using commercially available exonucleases or endonucleases according to standard procedures. For example, enzymes such as Bal31 or site-directed mutagenesis can be used to systematically cut off nucleotides from the ends of these genes. Also, genes that encode active fragments may be obtained using a variety of restriction enzymes. Proteases may be used to directly obtain active fragments of these proteins.

As discussed throughout, it is within the scope of the invention as disclosed herein that TSPs may be truncated and still retain functional/binding activity. By “truncated protein” it is meant that a portion of a protein may be cleaved and yet still exhibit binding activity after cleavage. Cleavage can be achieved by proteases, for example. Furthermore, effectively cleaved proteins can be produced using molecular biology techniques wherein the DNA bases encoding said protein are removed either through digestion with restriction endonucleases or other techniques available to the skilled artisan. For example, PCR can be used to make truncated proteins. After truncation, said proteins can be expressed in heterologous systems such as Escherichia coil, baculoviruses, plant-based viral systems, yeast and the like.

Because of the degeneracy/redundancy of the genetic code, a variety of different DNA sequences can encode the amino acid sequences disclosed herein. It is well within the skill of a person trained in the art to create alternative DNA sequences that encode the same, or essentially the same, proteins. These variant DNA sequences are within the scope of the subject invention.

The subject invention include, for example:
1) proteins obtained from wild type organisms;
2) variants arising from mutations;
3) variants designed by making conservative amino acid substitutions; and
4) variants produced by random fragmentation and reassembly of a plurality of different sequences that encode the subject proteins (DNA shuffling). See e.g. U.S. Pat. No. 5,605,793.

The DNA sequences encoding the subject proteins can be wild type sequences, mutant sequences, or synthetic sequences designed to express a predetermined protein. DNA sequences designed to be highly expressed in plants by, for example, avoiding polyadenylation signals, and using plant preferred codons, are particularly useful.

Certain proteins and genes have been specifically exemplified herein. As these proteins and genes are merely exemplary, it should be readily apparent that the subject invention comprises use of variant or equivalent proteins (and nucleotide sequences coding for equivalents thereof) having the same or similar functionality as the exemplified proteins. Equivalent proteins will have amino acid similarity (and/or homology) with an exemplified protein. Preferred polynucleotides and proteins of the subject invention can be defined in terms of narrower identity and/or similarity ranges. For example, the identity and/or similarity of the TSP/PRBP/PRBD can be 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified or suggested herein.

Unless otherwise specified, as used herein, percent sequence identity and/or similarity of two nucleic acids is detennined using the algorithm of Karlin and Altschul (1990), Proc. Natl. Acad. Sci. USA 87:2264-2268, modified as in Karlin and Altschul (1993), Proc. Natl. Acad. Sci. USA 90:5873-5877. Such an algorithm is incorporated into the NBLAST and XBLAST programs of Altschul et al. (1990), J. Mol. Biol. 215:402-410. BLAST nucleotide searches are performed with the NBLAST program, score=100, wordlength=12. Gapped BLAST can be used as described in Altschul et al. (1997), Nucl. Acids Res. 25:3389-3402. When utilizing BLAST and Gapped BLAST programs, the default parameters of the respective programs (NBLAST and XBLAST) are used. See NCBI/NIH website. The scores can also be calculated using the methods and algorithms of Crickmore et al. as described in the Background section, above.

To obtain gapped alignments for comparison purposes, the AlignX function of Vector NTI Suite 8 (InforMax, Inc., North Bethesda, Md., U.S.A.), was used employing the default parameters. These were: a Gap opening penalty of 15, a Gap extension penalty of 6.66, and a Gap separation penalty range of 8. Two or more sequences can be aligned and compared in this manner or using other techniques that are well-known in the art. By analyzing such alignments, relatively conserved and non-conserved areas of the subject polypeptides can be identified. This can be useful for, for example, assessing whether changing a polypeptide sequence by modifying or substituting one or more amino acid residues can be expected to be tolerated.

The amino acid homology/similarity/identity will typically {but not necessarily) be highest in regions of the protein that account for its activity (e.g. binding activity) or that are involved in the determination of three-dimensional configurations that are ultimately responsible for the activity. In this regard, certain amino acid substitutions are acceptable and can be expected to be tolerated. For example, these substitutions can be in regions of the protein that are not critical to activity. Analyzing the crystal structure of a protein, and software-based protein structure modeling, can be used to identify regions of a protein that can be modified (using site-directed mutagenesis, shuffling, etc.) to actually change the properties and/or increase the functionality of the protein.

Various properties and three-dimensional features of the protein can also be changed without adversely affecting the binding activity/functionality of the protein. Conservative amino acid substitutions can be expected to be tolerated/to not adversely affect the three-dimensional configuration of the molecule. Amino acids can be placed in the following classes: non-polar, uncharged polar, basic, and acidic. Conservative substitutions whereby an amino acid of one class is replaced with another amino acid of the same type fall within the scope of the subject invention so long as the substitution is not adverse to the biological activity of the compound. Amino acids belonging to each class are as follows:

Classes of amino acids.

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

In some instances, non-conservative substitutions can also be made. The critical factor is that these substitutions must not significantly detract from the functional/biological activity of the protein.

Equivalent proteins and/or genes encoding these equivalent proteins can be obtained/derived from wild-type or recombinant bacteria, for example.

Variant genes can be used to produce variant proteins; recombinant hosts can be used to produce the variant proteins. Using these “gene shuffling” techniques, equivalent genes and proteins can be constructed that comprise a range of contiguous residues (amino acid or nucleotide) of any sequence exemplified herein, the potential sizes of which are provided in more detail below. Thus, fragments for use in gene shuffling techniques, and fragments of

TSPs to be used directly, can comprise a range of contiguous residues of any protein exemplified or suggested herein, said fragments comprising, for example, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 contiguous residues (amino acid or nucleotide), for example (and if appropriate), corresponding to a segment (of the same size) in any of the exemplified or suggested sequences (or the complements (full complements) thereof). Similarly sized segments, especially those for conserved regions, can also be used as probes and/or primers.

Unless specifically indicated or implied, the terms “a”, “an”, and “the” signify “at least one” as used herein.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

### Methods for TSP Characterization

### Materials and Methods

Salmonella typhimurium (ATCC19585), Staphylococcus aureus (ATCC12598) and P22 phage (ATCC19585-B1) were purchased from American Type Culture Collection (Manassas, VA.). pET1 la expression vector and E. colI strain BL21(DE3) (expression host) were purchased from Novagen (Madison, Wis.).

1.1 Cloning and Expression of P22sTsps

Truncated versions of P22 phage tail spike gene lacking the codons for the first 108 amino acids (P22sTsp) were generated by a standard PCR using the phage P22 genome as the template. The primers incorporated Nde I and Bgl II sites as well as N- or C-terminal His₆ tags. The PCR products were cloned into pET11a vector followed by transformation in the E. coli strain BL21(DE3), using standard cloning techniques. Positive clones were identified by colony PCR and DNA sequencing.

The enzyme mutants P22sTsp5^{-x} (SEQ ID NO:4) and P22sTspH5^{-x} (SEQ ID NO:2) were constructed by splice overlap extention (SOE) and polymerase chain reaction (PCR) using, respectively, P22sTsp5 (SEQ ID NO:8) and P22sTsp5H (SEQ ID NO:6) genes within the pET11a vector. See Yau, K. Y. et al., 2005; “Affinity maturation of a V_{H}H by mutational hotspot randomization”; J Immunol Methods 297:213-224); and Ho, S. N., Hunt, H. D., Horton, R. M., Pullen, J. K., and Pease, L. R. (1989) Gene 77, 51-59.

In each ease, mutagenic primers were used to amplify two fragments which had an Asn instead of an Asp at position 392. The two fragments were then spliced together by SOE, amplified again by PCR and cloned for expression as described for the wild types. Vent DNA polymerase was used for PCR amplification to avoid incorporating errors into genes.

For expression, a single colony was used to inoculate 25 mL of LB medium (Sambrook, J. et al., 1989) containing 100 μg/mL ampicilin (LB/Amp) in a 100 mL Erlenmeyer flask. The flask was shaken overnight at 37° C. and 250 rpm. In the morning, 20 mL of the grown cell culture was used to inoculate 1 L of LB/Amp and the cells were incubated at 28° C. and 250 rpm until the cell density reached an OD₆₀₀ of ˜0.6. To induce protein expression, IPTG (isopropyl-beta-D-thiogalactopyranoside) was added to a final concentration of 400 pM and the cells were incubated at 30° C. and 250 rpm for 2 h.

The cells were pelleted by centrifuging the cultures at 8,000 g for 7 min at 4° C. and were subsequently re-suspended in 100 mL ice-cold lysis buffer (50 mM Tris-HCl pH 8.0, 25 mM NaCl, 2 mM EDTA) and stored at −20° C. overnight. The frozen suspensions were thawed at room temperature and immediately supplemented with PMSF (phenylmethylsulphonyl fluoride, 1 mM final concentration from 100 mM stock in ethanol) and DTT (dithiothreitol, 2 mM final concentration from 1 M aqueous solution).

The cells were lysed by adding freshly-prepared lysozyme (100 μg/mL final concentration from 2 mg/mL in 10 mM Tris-HCl buffer pH 8.0). The suspension was incubated at room temperature for 30 min with occasional mixing.

Subsequently, DNase I (Sigma-Aldrich Canada Ltd., Oakville, ON, Canada) (25 μg/mL final concentration from 0.5 mg/mL stock in 0.5 M MgCl₂) was added and the lysate was incubated at room temperature for 30 min. The lysate was centrifuged at 14,000 g for 5 min at 4° C. and the supernatant was dialyzed against 10 mM HEPES (N-[2-hydroxyethyllpiperazine-N′42-ethanesulfonic acid]) buffer pH 7.0, 500 mM NaCl and subjected to protein purification by immobilized metal affinity chromatography (IMAC) as described (Tanha, J. et al., 2003).

Protein concentrations were determined by A₂₅₀ measurements using molar absorption coefficients calculated for each protein (Pace, C. N. et aL, 1995). Size exclusion chromatography was performed on Superdex 200 columns (GE Healthcare, Baie d'urfe, QC, Canada). Protein expression was monitored by Western blotting using an anti-Hisₛ antibody (QIAGEN, Mississauga., ON, Canada) as the primary antibody against aliquots taken at various stages during extraction.

### 1.2. Growth of Bacteria

Fifteen mL of nutrient broth, NB (5 g peptone and 3 g meat extract in 1 L water, pH 7.0) was inoculated with a single S. typhimurium colony from a NB plate (Sambrook, J. et al., 1989). The bacteria were grown overnight at 37° C. at 200 rpm. In the morning, the culture was spun down in Eppendorf tubes with a microfuge at maximum speed for 30 s, the supernatant was removed and the cell pellet was re-suspended in 10 mL PBS buffer. The cells were re-spun, the supernatant was removed and the cell pellet was re-suspended in 10 mL PBS buffer. Cell density was measured at OD₆₀₀ using diluted samples (1 OD₆₀₀=1×10⁸ cells/mL). S. aureus was grown as described for S. typhimurium but in brain heart infusion media (EMD Chemicals Inc., Darmstadt, Germany). Cells were used in enzyme-linked immunosorbent assays (ELISA) or micro-agglutination assays.

To prepare cells for in vivo experiments (Subsection 2.1), a frozen stock of Salmonella was streaked on a Xylose Lysine Desoxycholate (XLD) plate (Cat No. MP2480, Oxoid Company, Nepean, ON, Canada) followed by incubation at 37° C. for 18-24 h. Three milliliter NB in a 15-mL falcon tube was inoculated with a single colony of Salmonella from the XLD plate. The tube was incubated in a shaker incubator at an angle for 18-24 h at 200 rpm and 37° C. (The overnight culture of Salmonella would typically have an OD₆₀₀ of about 1.4-1.6.) A 1:100 dilution of the cells in 2×10 mL NB in 50 mL Falcon tubes was made and the cells were grown at 200 rpm and 37° C. until an OD₆₀₀ of 0.3-0.5 was reached (˜2-3 h). Cells were centrifuged at 12,000 rpm in a microfuge for 1.5 min and re-suspended in PBS for a final OD₆₀₀ of 1.0. Titerations, on XLD plates, were also performed to confirm the cell density. Cells were immediately used to orally inoculate chicks (see Subsection 2.1).

### 1.3. ELISA

Microtiter wells were coated overnight with 100 μL of 5 μg/mL Salmonella O-antigen-specific antibody Se155 IgG (Sigurskjold, B. W. et al., 1991) in PBS at 4° C. The microtiter wells were emptied, blotted on a paper towel, filled with 300 μL of 1% CPBS (1% casein in PBS), covered and incubated for 2 h at 37° C. for blocking. The wells were emptied, blotted and added with 50 μL of Salmonella cells (OD₆₀₀=1) and 50 μL of 2% CPBS. The contents were mixed and incubated at room temperature for 1 h. The wells were emptied, blotted and washed 5× with cold (4° C.) PBST (PBS/0.05% Tween 20).

Serial two-fold dilution of P22sTsp in 100 μL cold 1% CPBS was added to wells 1-23. To well 24, 100 μL 1% CPBS was added. The wells were incubated on ice for 1 h. The wells were washed with cold PBST, 100 μL cold rabbit anti-P22sTsp polyclonal antibodies (1/8,000 dilution) in 1% CPBS was added and the wells were incubated on ice for 1 h. The wells were emptied, blotted and washed again with cold PBST. Hundred microliters (1/5,000 dilution) HRP/anti-rabbit IgG monoclonal conjugate (Cedarlane Laboratories Ltd., Burlington, ON, Canada) in cold 1% CPBS was added to the wells followed by incubation on ice for 1 h. The wells were washed with cold PBST and 100 μL of a 1:1 ratio of peroxidase B reagent and TMB Peroxidase A reagent (KPL, Inc., Gaithersburg, Md.) was added. Following incubation for several minutes at room temperature, 100 μL of 1 M phosphoric acid was added to stop the reaction. Absorbance was read at 450 nm.

### 1.4. Micro-Agglutination Assay

Two fold dilutions of P22sTsp or Se155 IgG in PBS (+/−20 mM DTT) were performed from wells 1 to 11 in a microtiter plate. Well 12 (blank) had only PBS (+/−20 mM DTT). The total volume in each well was 50 μL. Subsequently, 50 μL of 1 OD₆₀₀ of S. typhimurium in PBS was added to all wells and the plate was incubated overnight at 4° C. or 42° C. In cell control experiments, S. aureus was used.

### 1.5. Preparation Offecal and Intestinal Protease Solution

Two grams of chick fecal matter was collected from the floor pen of chicks and re-suspended in 20 mL of sterile PBS by vigorous vortexing. The sample was centrifuged at 1,000 g on a swinging bucket centrifuge for 15 min at 4° C. One mL aliquots of the supernatant were transferred to microfuge tubes and spun at maximum speed in a microfuge to pellet down any remaining debris. The supernatants were collected and stored frozen in small aliquots at −20° C. for future digestion experiments. To prepare the intestinal tract protease solutions, four chicks were sacrificed and the intestinal contents were squeezed out of the intestinal tract and weighed. The appropriate amount of sterile PBS was added to make a 10-fold dilution. The samples were then processed and stored frozen as above.

### 1.6. Protease Experiments

Freshly-prepared 0.012 μg/μL sequencing grade trypsin or chymotrypsin (Hoffmann-La Roche Ltd., Mississauga, ON, Canada) in 1 mM HCl were used for digestion experiments. One μL of trypsin or chymotrypsin was mixed with 8 μL of 0.3 μg/μL P22sTsp in 100 mM Tris-HCl buffer pH 7.8 (trypsin) or in 50 mM Tris-HCl buffer plus 20 mM CaCl₂ (chymotrypsin). Reactions were carried out in a total volume of 10 μL for up to 1 h at 37° C. and stopped by adding 1 μL of 0.1 μg/μL trypsin/chymotrypsin inhibitor (Sigma-Aldrich Canada Ltd.). Following completion of digestion, samples were mixed with reducing SDS-PAGE loading buffer, boiled for 5 min at 95° C. and analyzed by PhastSystem SDS-PAGE apparatus according to the manufacturer's instructions (GE Healthcare). Pepsin digestion mixtures contained 8 μL of 0.3 μg/μL P22sTsp, 1 μL of 100 mM HCl and 1 μL of 0.012 μg/μL pepsin (Sigma-Aldrich Canada Ltd.). Reactions were carried out at 37° C. for up to 1 h and were subsequently analyzed by a reducing SDS-PAGE as described above.

To carry out digestion experiments with chick fecal and intestinal protease solutions, frozen stocks of protease solutions were thawed, diluted 10-fold and 30 μL of the diluted samples was mixed with 50 μg of P22sTsp5 (SEQ ID NO:8). Reactions were carried out in PBS buffer in a total volume of 80 μL at 37° C. for up to 2 h. The reactions were stopped by immediately boiling the samples for 5 min. In protease-negative experiments, 30 μL of inactivated protease solutions were used (inactivated samples were prepared by heating chick fecal or intestinal tract protease solutions at 95° C. for 15 min followed by cooling down on ice). In P22sTsp-negative control experiments, P22sTsps were replaced with single domain antibody constructs (sdAb). Following completion of digestions, aliquots were removed and analyzed by a reducing SDS-PAGE.

### [0 1.7. Results for TSP In Vitro Characterization

1.7.1. Cloning and Expression of P22sTsps

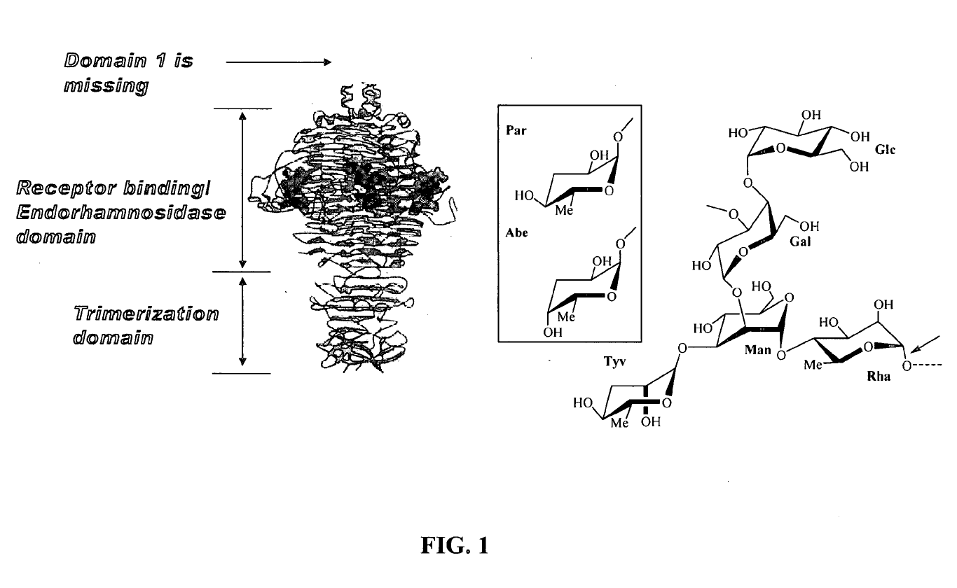

A truncated version of P22 tail spike protein (P22sTsp) spanning residues 109-666 was amplified out of P22 phage genome by PCR {FIGS. 1 & 2A). The PCR step also added the codons for a His₆ tag for subsequent protein purification by IMAC. The construct was cloned into the pET11a expression vector which further added RSGC at the C-terminus of P22sTsp. The Cys residue was included to cause hexamer formation through inter-trimer disulfide linkages. Five positive clones were identified by colony PCR and sequenced. All had mutations with respect to a deposited reference sequence (protein accession No.: AAF75060) (SEQ ID NO:9); mutations ranged from 5-7 amino acids {Table 1).

TABLE 1
Mutations identified in five P22sTsp clones

| | | | | | | |
|---|---|---|---|---|---|---|
| P22sTsp3 | N520H | | K561R | | A584P | Y590W |
| | S599N | | | | | |
| P22sTsp5* | | | K561R | G582V | A584P | Y590W |
| | S599N | | | | | |
| P22sTsp6 | N520H | | K561R | G582V | A584P | Y590W |
| | S599N | | | | | |
| P22sTsp18 | N520H | S521T | K561R | G582V | A584P | Y590W |
| | S599N | | | | | |
| P22sTsp20 | N520H | | K561R | G582V | A584P | Y590W |
| | S599N | | | | | |

The mutations are relative to the deposited Salmonella P22 phage Tsp sequence with protein accession No.: AAF75060;
*= (SEQ ID NO: 8)

The occurrence of mutations is non-random both in terms of amino acid identity and location. All the mutations are in the trimerization domain. However, the exact identical mutations were also observed at positions 582, 584, 590 and 599 for S. typhimurium bacteriophage ST64T Tsp (protein accession no. AAL15537) (SEQ ID NO:10), a Tsp which differs from P22 Tsp only at nine positions. P22sTsp3 and P22sTsp5 (SEQ ID NO:8) (FIG. 2B) which had the least number of mutations were chosen for expression employing pET11a/BL21(DE3) system. Following expression, proteins were purified by IMAC and yields up to 25 mg of purified protein per liter of bacterial culture were obtained.

1.7.2. Size Exclusion Chromatography and SDS-PAGE of P22sTsps

Following purification, P22sTsp3 and P22sTsp5 (SEQ ID NO:8) were analyzed by size exclusion chromatography (SEC) and SDS-PAGE. In SEC, both proteins had the same profile and each gave two peaks. (Size exclusion chromatograms and SDS-PAGE profiles were produced and observed for non-reduced and reduced P22sTsps, for example. One peak corresponded to the hexameric P22sTsp and the other peak corresponded to the trimeric P22sTsp. The following were observed: non-reduced P22sTsp5 (SEQ ID NO:8); reduced P22sTsp5 (SEQ ID NO:8); non-reduced P22sTsp5^{31 x} (SEQ ID NO:4); and reduced P22sTsp5^{31 x} (SEQ ID NO:4).) From a graph of logMW versus elution volume obtained from standard protein mass markers chromatogram, apparent molecular masses of 210 kDa and 400 kDa were obtained for the two peaks. These are very close to the expected molecular masses for the trimeric (184 kDa) and hexameric (368 kDa) P22sTsp. The slightly higher than expected molecular mass have been reported before for P22 phage Tsp as well as for Shigella phage Sf6 Tsp and attributed to their elongated shapes (Freiberg, A. et al., 2003). The ratio of the hexamer to trimer was different from batch to batch ranging from 1.5:1 to 3:1.

In subsequent experiments, P22sTsp3 and P22sTsp5 (SEQ ID NO:8) were incubated in 20 mM DTT at room temperature for 30 min and then subjected to SEC. Compared to the non-treated proteins, the reduced ones had their hexamers converted to trimers, indicating that the hexamers, as expected, are formed from trimers by disulfide linkages.

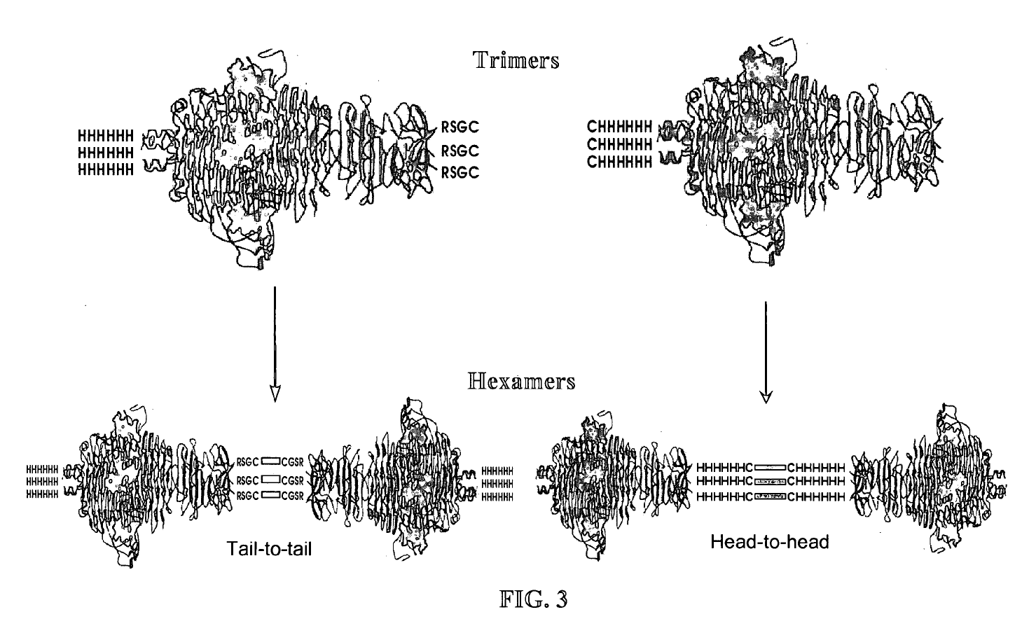

Subsequently, denaturing SDS-PAGE of non-reduced and reduced P22sTsp3 and P22sTsp5 (SEQ ID NO:8) were performed. In the non-reduced states, both proteins gave two bands with corresponding apparent molecular masses of 61.9 and 107 kDa as determined from a standard curve of logMW versus mobility. The molecular mass of the “61.9 kDa band” is very close to the theoretical molecular mass for the monomeric P22sTsp polypepₜide chain (61.3 kDa) one would expect on a denaturing SDS-PAGE _{g}el. Upon, reduction the 107 kDa band is converted to the 61.3 kDa band, demonstrating that it corresponds to a dimeric chain formed by disulfide linkage between two monomeric chains. The SDS-PAGE results are, therefore, consistent with the size exclusion chromatpgraphy data, which showed that the hexamers are formed from trimers by disulfide linkages. One would expect that, in addition to generating monomeric polypeptide chains on SDS-PAGE gels, the hexamers also generate dimeric chains-monomers linked by a disulfide linkage. The recombinant hexameric (tail-to-tail) construct as well as its parental trimeric construct is shown in FIG. 3.

1.7.3. ELISA and Micro-Agglutination Assay of P22sTsps

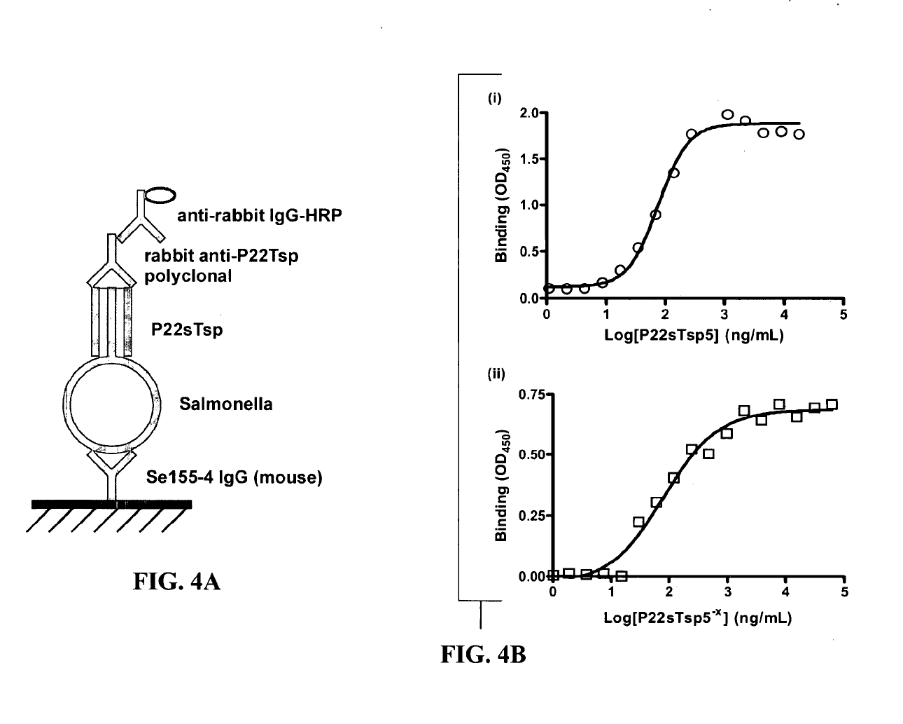

We performed ELISA according to the scheme in FIG. 4A to assess the binding of P22sTsp5 (SEQ ID NO:8) to Salmonella. Cells were captured on microtiter wells coated with Se155-4 mouse IgG and P22sTsp5 (SEQ ID NO:8) hexamer or trimer (purified by size exclusion chromatography) was added. Rabit Anti-sTsp polyclonal was added followed by the addition of anti-rabit IgG-HRP conjugate to detect binding. All the reagents and incubations were at 4° C. to quench the enzymatic activity of the P22sTsp5 (SEQ ID NO:8). P22sTsp trimer and hexamer bound to Salmonella cells with the same strength (50% binding value: 70 ng/mL) (FIG. 4Bi; data shown for the hexamer only). Post-ELISA SEC analysis of the P22sTsp preps used for the assays showed no trimer/hexamer inter-conversion.

The cell agglutination capability of P22sTsps was assessed by micro-agglutination assays. Two-fold serial dilutions of P22sTsps were added to round-bottom microtiter wells containing a constant number of Salmonella cells, leaving the last well without P22sTsp. The wells were incubated overnight at 4° C. or 42° C. In a micro-agglutination assay, agglutinated cells appear diffused whereas the non-agglutinated ones appear as round dots. (P22sTsp micro-agglutination assays were conducted and observed at 4° C. and 42° C. Minimal agglutination concentration values are recorded in Table 2. No agglutination was observed with S. aureus at the highest concentration used.). The minimum concentration of P22sTsp which resulted in detectable cell agglutination (minimum agglutination concentration, MAC) was used as a measure of agglutination potency of P22sTsp. Initial micro-agglutination assays at 4° C. revealed that P22sTsp3 and P22sTsp5 (SEQ ID NO:8) were indistinguishable in terms of their ability to agglutinate Salmonella. Thus, all subsequent experiments were performed with P22sTsp5 (SEQ ID NO:8). At 4° C., both the trimeric and hexameric versions of P22sTsp5 (SEQ ID NO:8), purified by size exclusion chromatography, agglutinated cells with the same strength (Table 2).

TABLE 2
Salmonella¹ microagglutination assay comparing
P22sTsp trimers and hexamers at 4° C.

Minimal agglutination
concentration (ng/mL)

| P22sTsp²/IgG | Trimer³ | Hexamer³ |
|---|---|---|
| P22sTsp5 (SEQ ID NO: 8) | 149 | 138 |
| P22sTsp5^{-x} (SEQ ID NO: 4) | 118 | 121 |
| P22sTsp5H (SEQ ID NO: 6) | 150 | 110 |
| P22sTsp5H^{-x} (SEQ ID NO: 2) | 76 | 133 |

| | |
|---|---|
| Se155-4 IgG⁴ | 320 |

¹Salmonella enterica serovar Ttyphimurium was used for the microagglutination assays.
² None of the P22sTsps showed agglutination against S. aureus.
³Trimer and hexamer P22sTsps5 were purified by size exclusion chromatography.
⁴Se155-4 is specific to the O-antigen on the surface of Salmonella typhimurium.

The MAC values were 149 ng/mL and 138 ng/mL for the trimer and hexamer, respectively, virtually indistinguishable. Post-agglutination SEC analysis of the P22sTsp preps used for agglutination showed no trimer/hexamer inter-conversion. Trimers obtained by reducing the hexamers with 20 mM DTT gave the same MAC value. In this case, all the wells had 10 mM DTT to prevent re-conversion of trimers to hexamers by oxidation. The hexamers show a slightly better agglutination capability than Se155-4 (Sigurskjold, B. W. eₜ al., 1991), an IgG which is specific to O-antigen of the Salmonella used in micro-agglutination assays (MAC=320 ng/mL).

The agglutination was specific since neither the trimeric nor the hexameric P22sTsp5 (SEQ ID NO:8) agglutinated Staphylococos aureus. However, no agglutination was observed at 42° C. (physiological temperature in chickens). Cell agglutination is essentially a binding event and this can be interfered by the enzymatic activity of the endorhamnosidase in the central domain of P22sTsp5 (SEQ ID NO:8). At 4° C., the central domain should act only as a binding domain and thus agglutination occurs. At 42° C., the central domain additionally acts as endorhamnosidase and, thus, interferes with the agglutination process. Thus, to have agglutination at 42° C., we would need a P22sTsp with intact receptor binding and defective endorhamnosidase activity.

1.7.4. Construction and Analysis of P22sTsp5^{-x} (SEQ ID NO:4)

We constructed an enzymatic mutant of P22sTsp5 (SEQ ID NO:8), termed P22sTsp5^{-x} (SEQ ID NO:4), which maintains the binding activity of the wild type. The mutant differs from the wild type only at position 392: D392N (Baxa, U. et al., 1996). As with the wild type, P22sTsp5^{31 x} (SEQ ID NO:4) also existed as a mixture of trimers and hexamers, and here too the hexamer was formed by disulfide-mediated linkage of two trimers. In ELISA, both trimer and hexamer bound to Salmonella cells with the same strength and gave the same 50% binding values as the wild types' (FIG. 4Bii; data shown for the trimer only). They also exhibited virtually the same agglutination strength at 4° C. with respect to each other and compared to the wild type trimer and hexamer (see the MAC values in Table 2).

Using unfractionated P22sTsp, we compared the agglutination potency of the mutant to the wild type at 4° C. and 42° C. At 4° C., P22sTsp5 (SEQ ID NO:8) and P22sTsp5^{-x} (SEQ ID NO:4) had MAC values which were almost the same (Table 3) and very similar to those for the fractionated P22sTsp (Table 2). At 42° C., however, P22sTsp5^{-x} (SEQ ID NO:4) had a MAC value of 12,900 ng/mL whereas P22sTsp5 (SEQ ID NO:8) did not show any agglutination at the highest concentration examined (400,000 ng/mL) (Table 3). This shows at least a 30-fold improvement in the MAC value and demonstrates that diminishing the enzymatic activity of the P22sTsp makes it a very effective agglutinator at the physiological temperature. However, the MAC value of P22sTsp5′ (SEQ ID NO:4) at 42° C. is 130-fold higher than that at 4° C. (12,900 ng/mL versus 100 ng/mL). This is most likely due to the fact that P22sTsp5^{-x} (SEQ ID NO:4) still has some residual enzymatic activity (Baxa, U. et al., 1996). The enzymatic activity of P22sTsp5^{-x} (SEQ ID NO:4) can further be reduced or eliminated by additional mutations in the active site (Baxa, U. et al., 1996). This should result in further improvement of the agglutination capability of P22sTsp5 (SEQ ID NO:8). Fractionated and unfractionated P22sTsp5^{-x} (SEQ ID NO:4) did not agglutinate S. aureus.

1.7.5. Construction and Analysis of P22sTsp5H (SEQ ID NO:6)and P22sTsp5H^{-x} (SEQ ID NO:2)

Previous two constructs generate hexamers in a tail-to-tail orientation. We also constructed the wild type and enzyme mutant P22sTsp with the Cys residue at their 5′ end: P22sTsp5H (SEQ ID NO:6) and P22sTsp5H^{-x} (SEQ ID NO:2), respectively. Similar to the previous two constructs, P22sTsp5H (SEQ ID NO:6) and P22sTsp5H^{-x} (SEQ ID NO:2) were expressed in high amount, and as mixtures of trimers and hexamers as was shown by reducing and non-reducing SEC and SDS-PAGE experiments. However, P22sTsp5H (SEQ ID NO:6) and P22sTsp5^{-x} (SEQ ID NO:2) hexamers are expected to exist in a head-to-head orientation (FIG. 3). Both constructs demonstrated the same protease resistance profile. At 4° C., both constructs had MAC values which were similar to each other and to those for P22sTsp5 (SEQ ID NO:8) and P22sTsp5^{-x} (SEQ ID NO:4) (Tables 2 and 3). As seen with P22sTsp5 (SEQ ID NO:8), the agglutination capability of P22sTsp5H (SEQ ID NO:6), which was nonexistent at 42° C. at the highest concentration used, was drastically improved upon D392N mutation in P22sTsp5H^{-x} (SEQ ID NO:2), by at least 80-fold (Table 3). Again, as with P22sTsp5^{-x} (SEQ ID NO:4), P22sTsp5H^{-x} (SEQ ID NO:2) also showed a much lower MAC value at 4° C. than at 42° C.: 63 ng/mL versus 2,030 ng/mL (Table 3). However, P22sTsp5^{-x} (SEQ ID NO:2) was over six times better an agglutinator than P22sTsp5^{-x} (SEQ ID NO:4) at 42° C. (see MAC values in Table 3).

TABLE 3
Salmonella¹ microagglutination assay at 4° C. and 42° C.

Minimal agglutination
concentration (ng/mL)

| P22sTsp² | 4° C. | 42° C. |
|---|---|---|
| P22sTsp5 (SEQ ID NO: 8) | 98 | >400,000 |
| P22sTsp5^{-x} (SEQ ID NO: 4) | 100 | 12,900 |
| P22sTsp5H (SEQ ID NO: 6) | 77 | >160,000 |
| P22sTsp5H^{-x} (SEQ ID NO: 2) | 63 | 2,030 |

¹Salmonella enterica serovar Ttyphimurium was used for the microagglutination assays.
² None of the P22sTsps showed agglutination against S. aureus.

### 1.7.6. Protease Studies

Protein therapeutics are more efficacious for GI tract applications if they are resistant to trypsin, chymotrypsin and pepsin. We investigated the degree of resistance of P22sTsp5 (SEQ ID NO:8) to aforementioned GI proteases at 42° C. P22sTsp was treated with trypsin for 1 h and was subsequently analyzed by SDS-PAGE and SEC. As shown by an SDS-PAGE gel, there was no quantitative difference between the undigested and the digested proteins, demonstrating that P22sTsp is completely resistant to trypsin. (This was observed by non-reducing SDS-PAGE and SEC analyses of trypsin-treated P22sTsp5 (SEQ ID NO:8). The SDS-PAGE gel comprised a molecular weight marker in lane 1; lane 2, untreated P22sTsp5 (SEQ ID NO:8); lane 3, trypsin-treated P22sTsp5 (SEQ ID NO:8) (1 h at 42° C.); lane 4, trypsin-treated VHH (positive control); lane 5, untreated VHH containing trypsin/chymotrypsin inhibitor; trypsin/chymotrypsin inhibitor; #, trypsin. The SEC analyses showed untreated P22sTsp5 (SEQ ID NO:8) and trypsin-treated P22sTsp5 (SEQ ID NO:8). This is consistent with the previously published results (Steinbacher, S. et al., 1994). However, the fact that the hexamer is not converted to a trimer demonstrates that the Arg residues in between and outside the two trimeric units are not accessible to trypsin (Trypsin cleaves C-terminal to Arg or Lys) (FIG. 3). A positive VHH protein control showed a complete digestion, demonstrating that pepsin was active. Consistent with the SDS-PAGE results, both trimeric and hexameric P22sTsp gave identical SEC profiles under digestion and non-digestion conditions at 42° C., demonstrating again that the protein was completely resistant to trypsin (per SEC analyses). However, P22sTsp5 (SEQ ID NO:8) was somewhat sensitive to chymotrypsin and sensitive to pepsin at physiological temperatures (SDS-PAGE analyses were conducted for chymotrypsin-treated and pepsin-treated P22sTsp5 (SEQ ID NO:8). For the chymoptrypsin analyses, Lane 1 comprised molecular weight markers; Lanes 2 and 3, chymotrypsin-treated pentameric VHH control (0 and 60 min incubation, respectively); Lanes 4, 5 and 6, chymotrypsin-treated P22sTsp5 (SEQ ID NO:8) (0, 20 and 60 min incubation, respectively). For the pepsin-treated analyses, Lane 1 comprised molecular weight markers; Lanes 2, 3, and 4, pepsin-treated P22sTsp5 (SEQ ID NO:8) (0, 20 and 60 min incubation, respectively). Locations of the control protein and P22sTsp5 (SEQ ID NO:8) were noted.

Next, P22sTsp5 (SEQ ID NO:8) was tested for its resistance to proteases in chicken feces and intestinal contents. P22sTsp5 (SEQ ID NO:8) was completely resistant to protease from both sources for up to 2 h at 37° C., whereas a control single domain antibody was completely digested. (Reducing SDS-PAGE analyses were performed for P22sTsp5 (SEQ ID NO:8) following treatment with chicken fecal and intestinal content proteases. Lane 1 comprised molecular weight markers; lane 2, untreated P22sTsp5 (SEQ ID NO:8) in PBS; lane 3-6, P22sTsp5 (SEQ ID NO:8) incubated with protease solutions at 37° C. for 5 min, 20 min, 1 h and 2 h, respectively; Lane 7, a sdAb control incubated with fecal protease solution at 37° C. for 2 h. The sdAb control incubated with intestinal protease solution also showed a complete digestion). The control protein was not digested when treated with heat-inactivated protease solutions for 2 h at 37° C.

Protease experiments were also performed for P22sTsp^{-x} (SEQ ID NO:4), P22sTsp5H (SEQ ID NO:6) and P22sTsp5H^{-x} (SEQ ID NO:2), and the results were the same as the ones obtained for P22sTsp5 (SEQ ID NO:8): all three P22sTsps were fully resistant to trypsin and proteases in chicken feces and intestinal contents, somewhat sensitive to chymotrypsin and sensitive to pepsin at physiological temperatures.

### EXAMPLE 2

### Methods for Reduction of Colonization of Bacteria in Poultry

### 2.1 In Vivo Experiments

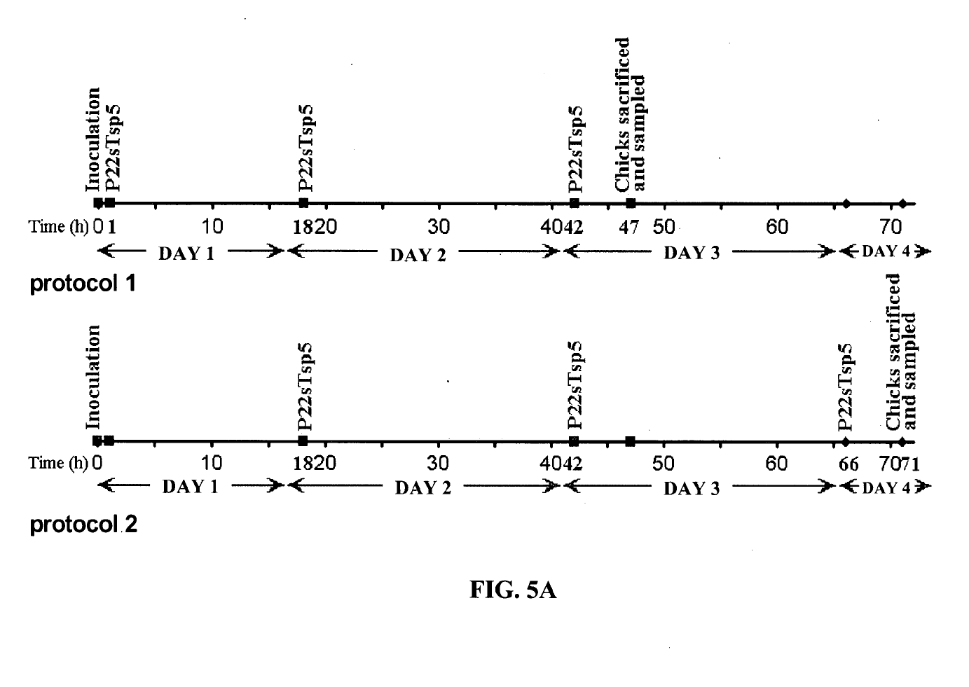

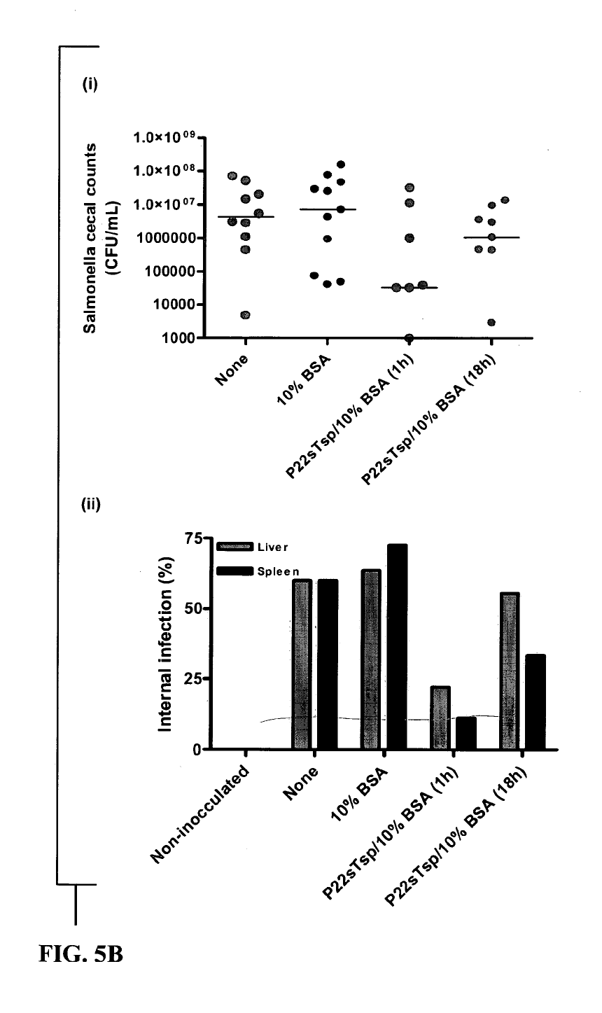

One-day-old chicks were arrived, acclimated and tagged on day 1. 10% of the chicks, selected at random, were swabbed cloacally with calcium alginate swabs (Cat. No. 14-959-77, Fisher Scientific, Ottawa, ON, Canada). The swabs were used to streak on XLD plates which were subsequently incubated at 37° C. overnight for determining the presence of endogenous Salmonella. (Following incubation, XLD will appear red/pink with Salmonella as black colonies.) On day 2, 2-day-old chicks were orally gavaged (time 0) with 10⁴-10⁷ Salmonella/300 μL PBS (see Subsection 1.2 for cell preparation). Chicks were subsequently gavaged with 30 μg/300 μL of P22sTsp5 (SEQ ID NO:8) at time 1, 18 and 42 h (Protocol 1) or 18, 42 and 66 h (Protocol 2) (FIG. 5A) Oral dose was gavaged by attaching a piece of Nalgene® tubing (Nalge Nunc International Corp, Rochester, N.Y.) with ⅛ ID×¼ OD× 1/16 wall to a 1-mL syringe, inserting the tubing into the crop, and gavaging 300 μL on the inoculate. Following the sacrifice, the cecal materials, spleens and livers were collected and processed for tittering as described below.

(a) Cecal material. The cecal samples were weighed (≦0.2 g), diluted 10× in PBS (10⁻¹ dilution) and vortexed. Further serial dilutions up to 10⁻⁶ were performed in PBS in total volumes of 200 μL in 96-well microtiter plates using a multi-channel pippetter. Starting from the highest dilution, the contents of the wells were pipetted up and down a few times and 50 μL was plated on XLD plates. The plates were let dry and subsequently incubated at 37° C. overnight. The titer of the colonies on serial dilution XLD plates was determined in the morning.

(b) Spleen and liver. To 2-mL screw cap tubes (Cat. No. SS12331-S0, Diamed, Mississauga, ON, Canada) two Bio 101 ¼″ ceramic beads (Q-Biogene, Carlsbad, Calif.) and 600 μL (for spleen) or 800 μL (for liver [right front lobe of liver was sampled]) of sterile PBS were added. Tubes were maintained on ice. Tubes were placed in Fastprep Instrument (Qbiogene, Carlsbad, Calif.) and processed at a speed of 4.0 for 2-5s (spleen) or 5-10 s (liver). Appropriate volumes of the homogenized samples which resulted in easily countable colonies (˜50 μL) were plated on XLD plates. Plates were incubated overnight at 37° C. and the titers were determined in the morning.

### 2.2 Results for Colony Reduction in Poultry—Animal Studies

We performed animal studies in chicks to determine to effect of orally administered P22sTsps on the colonization of Salmonella in the gut. Two-day-old chicks were infected with Salmonella followed by gavaging them with P22sTsp5 (SEQ ID NO:8). Three gavages were given in total and following the last one, birds were sacrificed and their ceca, spleen and liver were processed for Salmonella titer determination. Initially two protocols were tested (FIG. 5A). In Protocol 1, chicks were gavaged immediately after inoculation (1 h) with P22sTsp5 (SEQ ID NO:8) in 10% BSA or with 10% BSA alone. The next two gavages were given at 18 h and 42 h. In Protocol 2, the first gavage was delayed by 17 hours, given at 18 h.

As shown in FIG. 5B(i), inoculated chicks receiving no treatment (none) or receiving 10% BSA have median values of 4.3×10⁶ and 7.4×10⁶, respectively. In contrast, chicks receiving P22sTsp5 (SEQ ID NO:8) (Protocol 1) have a median of 3.3×10⁴, i.e., over 220-fold reduction in colonization compared to those receiving 10% BSA. For chicks receiving their first P22sTsp5 (SEQ ID NO:8) dose with 17 h delay (Protocol 2), the reduction in colonization was low (median=1.1×10⁶).

These results indicate that P22sTsp treatment prevents colonization but does not decolonize under the conditions described. Non-infected chicks had no Salmonella in their cecal contents. FIG. 5B(ii) shows the effect of orally administered P22sTsp5 (SEQ ID NO:8) on liver and spleen spread. Liver and spleen spread was also reduced in treated birds and correlates with cecal colonization data.

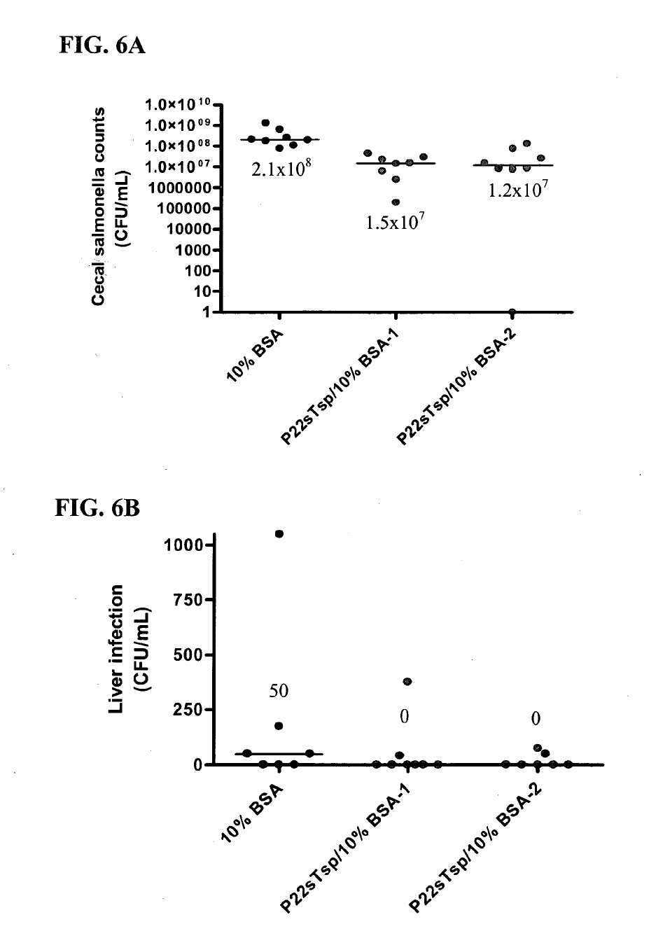

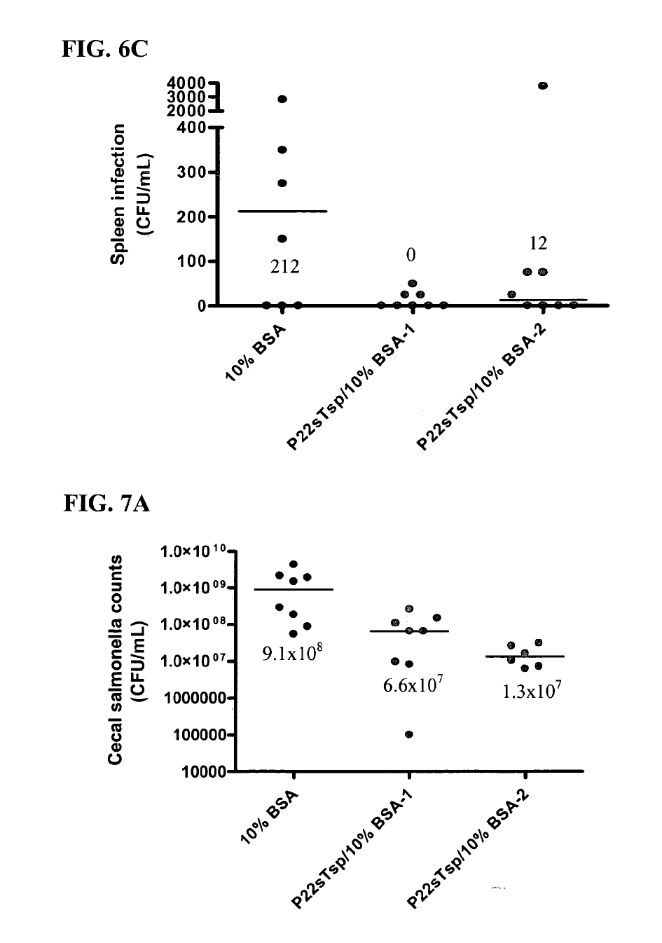

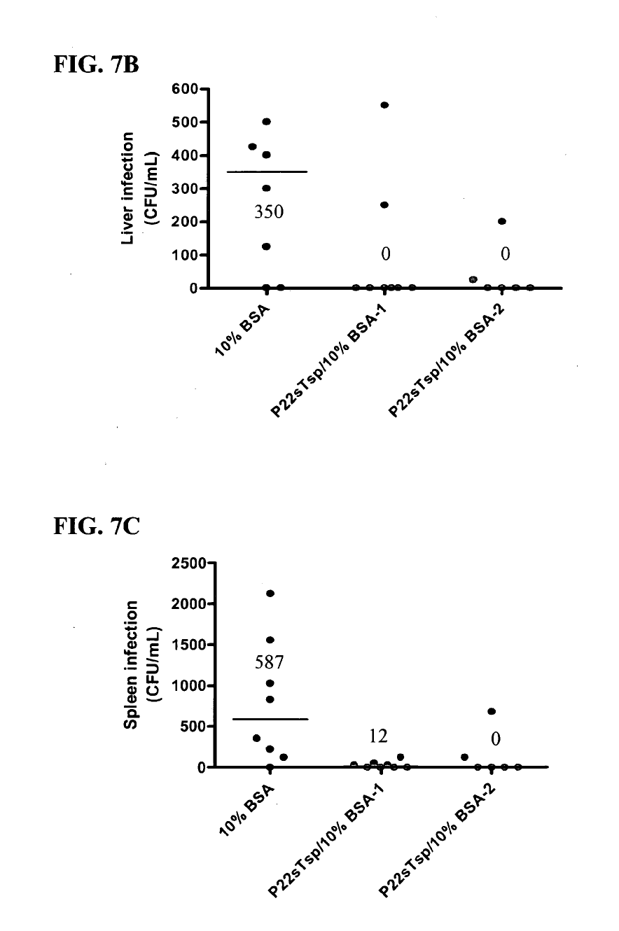

Experiments were repeated two more times with lower inoculation dose (FIGS. 6 and 7), and in each case the P22sTsp treatment group was done in duplicate (shown by numbers 1 and 2). In both cases Protocol 1 was followed. Again, oral administration of P22sTsp5 (SEQ ID NO:8) significantly reduced Salmonella colonization in ceca and infection in liver and spleen (see the median values on the graphs). Reduction in colonization was in the order of one to two orders of magnitude. Non-inoculated chicks had no bacteria detected in their ceca.

### EXAMPLE 3

### Motility Assay

Motility plates (NB plates/0.4% agar with or without 25 μg/ml filter-sterilized P22sTsp5 trimers) were made the day before their use and left at room temperature. P22sTsp5 (SEQ ID NO:8) and P22sTsp5^{-x} (SEQ ID NO:4) trimers were purified by size exclusion chromatography (Superdex 200™ column, GE Healthcare) using PBS as the equilibration buffer and added to the molten motility media just before pouring them into plates (50° C.). To perform motility assays, Salmonella cells were grown on NB plates overnight at 3TC (16-18 h). They were subsequently suspended in sterile PBS at a cell density of 1 OD₆₀₀. Employing a 10-μL pipettor, 5 μL of the cells were used to inoculate the centre of the motility plates, lightly piercing the surface of the agar plate with the pipettor tip; the plates were left unmoved until inoculation spots became dried. The plates were incubated up-side up at 37° C.

At different time points, the dimensions of the Salmonella spreads were measured to calculate their corresponding areas, which were used as a measure of the motility of cells. In control experiments, an equal volume of PBS replaced P22sTsps.

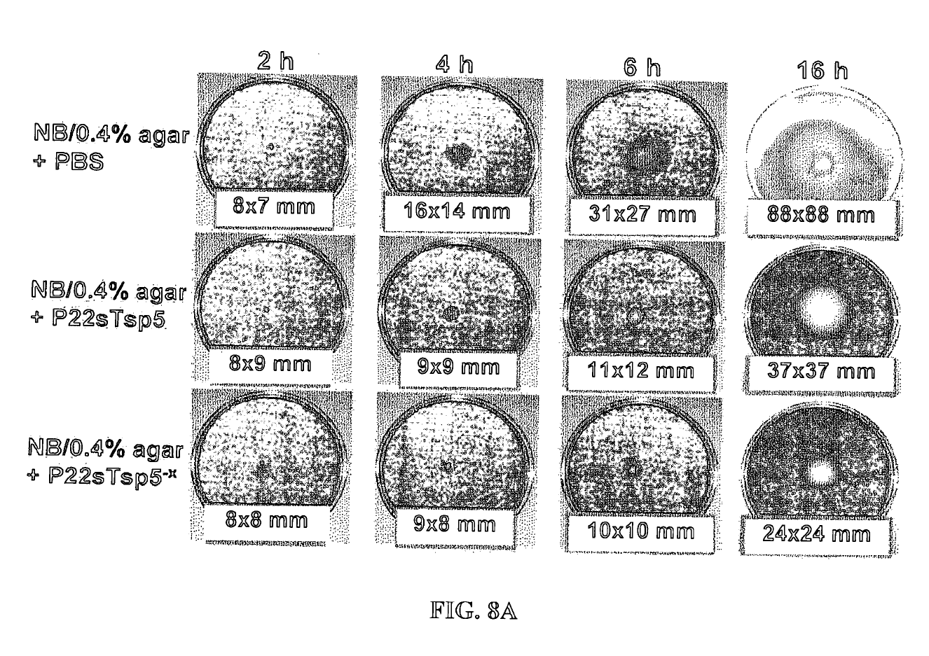

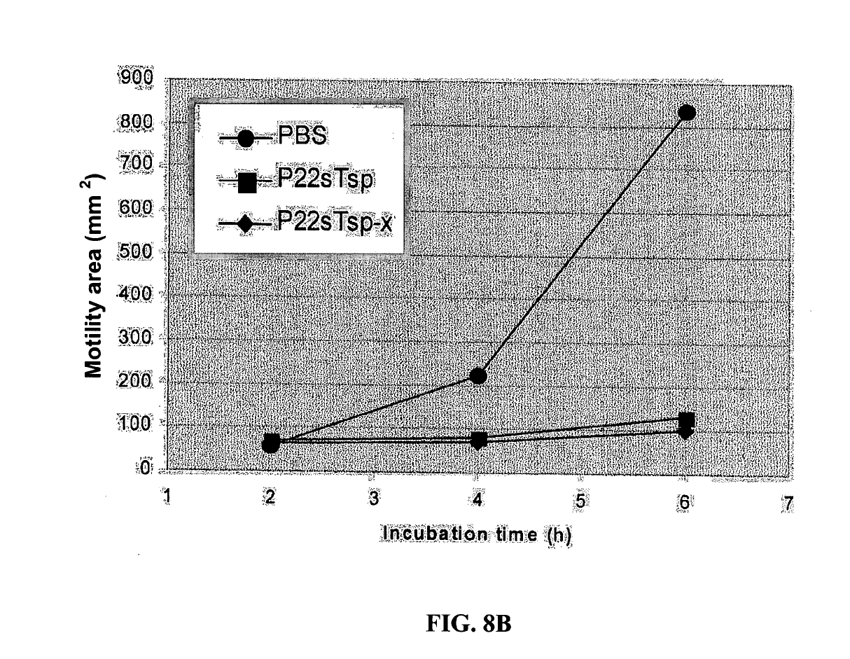

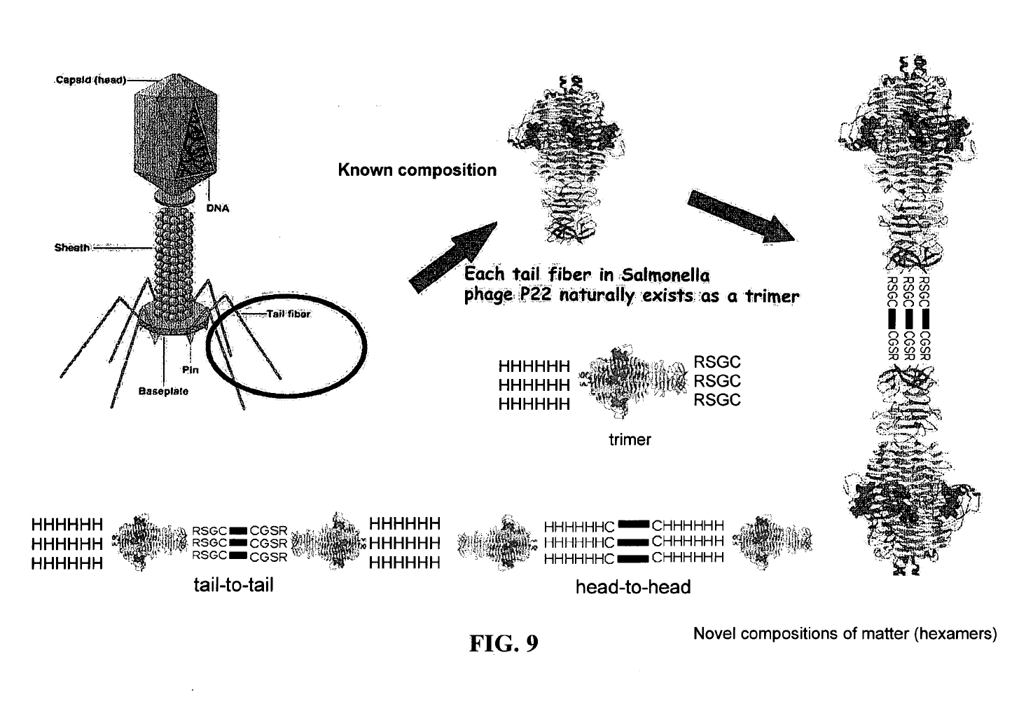

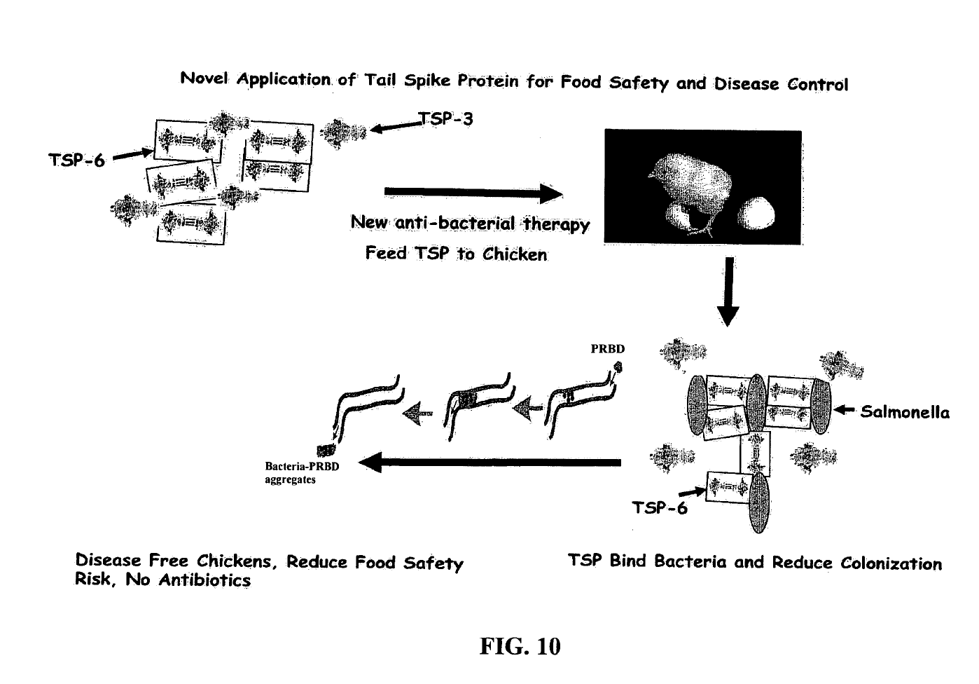

As can be seen in FIG. 8, the motility of Salmonella is significantly retarded in the presence of P22sTsp5 (SEQ ID NO:8) or P22sTsp5^{-x} (SEQ ID NO:4) in motility plates. FIG. 8A shows the dimensions of the Salmonella spreads on motility plates, which were measured at different time points and used to calculate motility areas. A graph of motility area versus incubation time was subsequently plotted (FIG. 8B).

Based on the fact that motility is a colonization factor for Salmonella (Siitonen et al. (1992) “Bacterial motility is a colonization factor in experimental urinary tract infection,” Infect.Immun. 60:3918-3920), which can be retarded by the LPS O-antigen-specific Tsps (as shown here), the use of Tsps of the subject invention can be used to prevent colonization in animals. That is, colonization can, at least in part, be mediated through the motility-retarding ability of Tsps. This is consistent with previous findings showing that compromising the structural integrity of LPS results in retarding Salmonella motility (Toguchi et al. (2000) “Genetics of swarming motility in Salmonella enterica serovar typhimurium: critical role for lipopolysaccharide,” J. Bacterial. 182: 6308-6321.

1. A method of administering an effective amount of an isolated phage receptor binding protein (PREP) to an animal, wherein said PRBP comprises a phage receptor binding domain (PRBD), and wherein said PRBD binds to a bacterial ligand on an outer membrane surface of a pathogenic bacterium.

2. The method of claim 1, wherein said said PRBP is an isolated bacteriophage tail spike protein TSP.

3. The method of claim 1, wherein said PRBP is a fragment of a bacteriophage tail spike protein (TSP).

4. The method of claim 1, wherein said ligand is selected from the group consisting of a carbohydrate and a protein.

5. The method of claim 1, wherein said bacteriophage is of the Order Caudovirales

6. The method of claim 1, wherein said bacteriophage is of the family Podoviridae.

7. The method of claim 1, wherein said pathogenic bacterium is an enteric bacterium.

8. The method of claim 1, wherein said pathogenic bacterium is Salmonella.

9. The method of claim 1, wherein said PRBP is injected into said animal.

10. The method of claim 1, wherein said PRBP is provided to said animal via animal feed.

11. The method of claim 1, wherein said animal is a vertebrate.

12. The method of claim 1, wherein said animal is selected from the group consisting of a human, a chicken, a swine, a bovine, a fish, a sheep, and a goat.

13. The method of claim 1, wherein said PRBP forms a homotrimer.

14. The method of claim 1, wherein said PRBP comprises an amino acid sequence selected from the group consisting of SEQ ID NO:2, SEQ ID NO:4, SEQ ID NO:6, and SEQ ID NO:8.

15. The method of claim 1, wherein said PRBP forms a homohexamer.

16. The method of claim 1, wherein said method reduces colonization of a pathogenic bacteria in a part of an animal selected from the group consisting of gut, mouth, and eyes.

17. A method of binding a surface ligand of a bacterium, said method comprising providing an isolated phage receptor binding protein (PRBP) comprising a phage receptor binding domain (PRBD), and contacting said PREP with a sample suspected of comprising said bacterium.

18. The method of claim 17, wherein said bacterium is a pathogenic bacterium.

19. The method of claim 17, wherein said sample is a water sample.

20. The method of claim 19, wherein said PREP is mounted on a filter.

21. The method of claim 17, wherein a disinfectant comprises said PRBP.

22. The method of claim 21, wherein said disinfectant is applied to a surface.

23. The method of claim 22, wherein said surface is selected from a surface of a slaughterhouse, a hospital, a medical device, a stent, and a catheter.

24. The method of claim 17, wherein said PRBP targets an undesired microbe in order to give a competitive advantage to a desired microbe.

25. The method of claim 24, wherein said desired microbe is involved in yogurt fermentation.

26. The method of claim 17, wherein said method prevents formation of industrial biofilm.

27. A pharmaceutical composition comprising an effective amount of at least one PRBP formulated for delivery to an animal's digestive tract.

28. A PRBP that assembles to form a homohexamer.
